(19) United States
(12) Patent Application Publication
(10) Pub. No.: US 2026/0263482 A1
(43) Pub. Date: Sep. 10, 2026

(54) COMPOSITION AND METHOD FOR INDUCING LUTEOLYSIS

(71) Applicant: WELFARE CONCEPTS LIMITED, Golflands, Auckland (NZ)

(72) Inventors: Ian George TUCKER, Golflands, Auckland (NZ); Prabhat BHUSAL, Golflands, Auckland (NZ); Greg WALKER, Golflands, Auckland (NZ); Scott MCDOUGALL, Golflands, Auckland (NZ); Forbes Richard EMSLIE, Golflands, Auckland (NZ)

(21) Appl. No.: 19/166,345

(22) PCT Filed: Apr. 9, 2024
(86) PCT No.: PCT/IB2024/053454

§ 371 Date: Sep. 17, 2025

(30) Foreign Application Priority Data
Apr. 18, 2023 (AU) 2023901142

(51) Int. Cl. A61K 31/5575; A61K 9/00; A61K 9/06; A61K 47/10; A61K 47/14; A61K 47/26

(52) U.S. Cl. CPC A61K 31/5575; A61K 9/0019; A61K 9/06; A61K 47/10; A61K 47/14; A61K 47/26

(57) ABSTRACT

An injectable sustained release composition comprising a prostaglandin, formulated to release the prostaglandin in a sustained manner, and a method of inducing luteolysis of a functional corpus luteum in an animal by the sustained administration of a prostaglandin such as PGF2α, preferably cloprostenol.

### RELATED APPLICATIONS

This application claims priority of Australian Patent Application No. 2023901142, filed 18 Apr. 2023, the entire contents of which are incorporated herein by way of reference.

### TECHNICAL FIELD

This invention relates, inter alia, to a method of inducing luteolysis in an animal as well as to inducing luteolysis of a functional corpus luteum in an animal by the sustained administration of a prostaglandin.

### BACKGROUND

In modern dairy production systems, reproductive performance is currently seen as one of the main targets for improving farm profitability, and is affected by many metabolic, physiological and immunological factors during the early post-partum period. Many reproductive performance indicators are used to evaluate cow fertility, but pregnancy rates are regarded as the most important indicator to ensure good farm profitability.

Considerable research has been conducted to develop reproductive protocols that ensure oestrus amongst cows within a herd are synchronized when required, and that reproductive rates are high. These reproductive protocols usually involve the introduction of hormones in various forms and in a staged manner. These treatments can take considerable time and money to implement, particularly when multiplied over hundreds of cows that are found in typical large herds.

One basic method of synchronizing oestrus is to administer a prostaglandin by injection to each cow in a herd, which is expected to result in around 75% of cycling animals to display oestrus in the following 2 to 5 days. This method is not effective for anoestrus cows, since a corpus luteum is not present in the ovary.

A more sophisticated protocol involves multiple interactions. For example, a treatment may start with the injection of an equivalent to endogenous gonadotropin releasing hormone (GnRH), which stimulates the maturation of ovarian follicles, ovulation and development of the corpus luteum.

In one version of a typical reproductive protocol, the next stage is to then inject the cow seven days later with an analogue of prostaglandin-F2 (PGF2α). This causes luteolysis (degradation) of the corpus luteum. Next the cows that appear to be in oestrus are mated. The cows that do not seem to be in oestrus are given another dose of GnRH and then inseminated shortly afterwards.

It has been demonstrated that a shorter period of follicular dominance can enhance embryo quality. Accordingly, a shorter protocol is wherein PGF2α is administered only five days after administration of GnRH has been proposed. Since this five-day protocol includes the administration of PGF2α at an earlier time compared to the more traditional seven day protocol, incomplete luteolysis can occur as some corpora lutea may not be fully responsive to the effects of PGF2α. This can lead to high progesterone concentrations around the second GnRH injection, reducing ovulation rates and consequently conception rates.

Recent studies suggest that a second administration of PGF2α at least 6 hours, but typically 24 hours, after the first injection of PGF2α can enhance luteolysis success by targeting corpora lutea which were refractory to treatment at the time of the first injection but which had developed receptors by the time of the second injection. However, the addition of a second administration of PGF2α adds to the complexity of the protocol and increases the handling of the animals.

There is a need in the art for improvements to the existing reproductive protocols in animals, particularly dairy cattle.

### DISCLOSURE OF THE INVENTION

It is an object of one or more embodiments of the present invention to address the foregoing problems, or at least to provide the public with a useful choice.

According to a first aspect of the present invention, there is provided a sustained release composition formulated for: inducing luteolysis of a functional corpus luteum in an animal; improving fertility in an animal; improving conception rates in a group of animals; synchronising ovulation in a group of animals; or, use in a reproductive breeding programme, wherein the composition comprises a prostaglandin.

According to a second aspect of the present invention, there is provided use of a prostaglandin in the manufacture of a medicament or composition for: inducing luteolysis of a functional corpus luteum in an animal; improving fertility in an animal; improving conception rates in a group of animals; synchronising ovulation in a group of animals; or, use in a reproductive breeding programme, wherein the medicament or composition provides a sustained release of the prostaglandin.

According to a third aspect of the present invention, there is provided a sustained release composition for use or when used in a method for: inducing luteolysis of a functional corpus luteum in an animal; improving fertility in an animal; improving conception rates in a group of animals; synchronising ovulation in a group of animals; or, use in a reproductive breeding programme, wherein the composition comprises a prostaglandin.

According to a fourth aspect of the present invention, there is provided a method of: inducing luteolysis of a functional corpus luteum in an animal; improving fertility in an animal; improving conception rates in a group of animals; or, synchronising ovulation in a group of animals; wherein the method comprises administering to the animal or animals a sustained release composition comprising a prostaglandin.

According to a fifth aspect of the present invention, there is provided use of a sustained release composition comprising a prostaglandin for: inducing luteolysis of a functional corpus luteum in an animal; improving fertility in an animal; improving conception rates in a group of animals; synchronising ovulation in a group of animals; or, use in a reproductive breeding programme.

According to a sixth aspect of the present invention, there is provided a kit for use in: inducing luteolysis of a functional corpus luteum in an animal; improving fertility in an animal; improving conception rates in a group of animals; synchronising ovulation in a group of animals; a reproductive breeding programme; or, controlling oestrus of an animal, wherein the kit comprises a sustained release composition comprising a prostaglandin.

According to a seventh aspect of the present invention, there is provided a kit for use in a method for controlling oestrus of an animal, wherein the kit comprises a sustained release composition of a prostaglandin.

According to an eighth aspect of the present invention, there is provided a method of preparing a sustained release composition of a prostaglandin, said method comprising the step of mixing a therapeutic quantity of the prostaglandin with a carrier that is capable of providing the prostaglandin with a sustained release such that the terminal half-life of the plasma concentration of the prostaglandin versus time profile is approximately 3.5 hours or more.

According to a ninth aspect of the present invention, there is provided an injectable sustained release composition comprising a prostaglandin, formulated to release the prostaglandin in a sustained manner.

According to a tenth aspect of the present invention, there is provided a sustained release composition comprising a prostaglandin and a carrier, formulated to release the prostaglandin in a sustained manner.

Features described below relate to all aspects of the invention described above, context permitting. Features described in respect of compositions/formulations/medicaments may also relate to methods/uses and vice-versa, context permitting. It is to be understood that the terms ‘formulation’, ‘medicament’ and ‘composition’ may be used interchangeably, context permitting.

### Prostaglandin

Any suitable type or types of prostaglandin can be used in the composition or in the methods/uses. Naturally occurring prostaglandins are 20-carbon unsaturated fatty acids that consist of a cyclopentane ring with two aliphatic side chains. Prostaglandins are structurally classified into nine major groups, A to I, each containing subgroups denoted by the subscripts 1, 2 and 3. These include prostaglandin E₁ (PGE₁), prostaglandin E₂ (PGE₂), prostaglandin F_{2α} (PGF_{2α}), prostacyclin (PGI₂), and thromboxane (TXA₂). In domestic animals, prostaglandin F2α (PGF_{2α} or PGF2α) is a particularly relevant prostaglandin to reproductive programmes.

When used herein (including in the claims), context permitting, the term ‘prostaglandin’ (‘PG’) refers to any prostaglandin or prostaglandin analogue, which is either naturally occurring or synthetically produced, and which has and/or exerts the desired characteristic in use, including pharmaceutically acceptable salts or esters thereof, or solvated forms thereof.

When used herein (including in the claims), context permitting, the term ‘PGF2α’ refers to Prostaglandin F2α, or an analogue thereof, including pharmaceutically acceptable salts or esters thereof, or solvated forms thereof. For instance, cloprostenol is a functional synthetic analogue of the naturally occurring PGF2α. For instance, dinoprost is naturally occurring PGF2α.

One of the known functions of PGF2α in the bovine oestrus cycle is to cause regression of the corpus luteum, which is a process known as luteolysis. The luteolysis of the corpus luteum results in a decrease in progesterone, which allows the dominant ovarian follicle to emerge from the last wave of follicles and mature. The dominant follicle is then able to produce sufficient oestrogen to cause oestrus.

A preferred prostaglandin for use in one or more compositions, medicaments, methods or uses of the invention is PGF2α. A preferred PGF2α is the synthetic analogue cloprostenol. A particularly preferred PGF2α is the sodium salt, cloprostenol sodium (anhydrous). Other potentially suitable salts include sodium cloprostenol hydrate, potassium cloprostenol, lithium cloprostenol and calcium cloprostenol, for example. A potentially suitable ester is isopropyl cloprostenol. Stearyl cloprostenol in an oily injection could have sustained action, as could other esters made using a fatty alcohol.

Another preferred PGF2α is dinoprost, which is naturally occurring PGF2α. A particularly preferred PGF2α is the dinoprost salt, dinoprost tromethamine.

When used herein (including in the claims), context permitting, the term ‘cloprostenol’ includes within its scope a solvated form of cloprostenol, as well as salts and esters thereof.

When used herein (including in the claims), context permitting, the term ‘dinoprost’ includes within its scope a solvated form of dinoprost, as well as salts and esters thereof.

### Sustained Release Composition

Preferably, the sustained release composition is formulated to provide the animal with a biologically active amount or concentration of prostaglandin over a sufficient period of time, for: inducing luteolysis of a functional corpus luteum in an animal; improving fertility in an animal; improving conception rates in a group of animals; synchronising ovulation in a group of animals; controlling oestrus of an animal; or, use in a reproductive breeding programme.

The phrase ‘to release the prostaglandin in a sustained manner’ and like expressions denote the sustained release composition's ability to release from the composition or from a carrier of the composition a biologically active concentration of prostaglandin for a particular period of time.

In some embodiments, the sustained release composition is formulated to provide a biologically active concentration of prostaglandin for approximately at least 8 hours after administration. In some embodiments, the sustained release composition is formulated to provide a biologically active concentration of prostaglandin for approximately at least 12 hours after administration. For example, the sustained release composition is formulated to provide a biologically active concentration of prostaglandin for approximately 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, or 24 hours after administration.

In some embodiments, the sustained release composition/medicament/formulation is capable of providing the prostaglandin with a sustained release such that the terminal half-life of the plasma concentration of the prostaglandin versus time profile is approximately 3.5 hours or more. (The terminal half-life is measured from the slope of the curve at times after the peak concentrations of drug in blood. For sustained release formulations when the absorption rate from the injection site is significantly slower that the elimination rate (the so-called flip-flop effect), the terminal half-life is mainly determined by the absorption rate.

Typically, the sustained release composition will be administered to the animal in an injectable form. That is, in some embodiments, the composition is formulated for injection, or administration of the composition to the animal comprises the step of injecting the animal with the composition. In some embodiments, the composition is injected subcutaneously. In some embodiments, the composition is injected intramuscularly.

In some embodiments, the sustained release composition is a liquid formulation. In some embodiments, the sustained release composition is a solution, suspension, dispersion, emulsion or (low viscosity) gel, suitable for injection. In some embodiments, the sustained release composition comprises a water-insoluble form of prostaglandin, such as a water-insoluble salt, and/or an encapsulated solid form of prostaglandin.

Typically, the sustained release composition comprises at least one type of carrier or a carrier that carries the prostaglandin. The carrier can comprise one or more ingredients.

Emulsions can be used to prolong the release of an active like prostaglandin by partitioning the active in one phase of the emulsion. For instance, an oil soluble active that is dissolved in the oil phase of an oil-in-water emulsion can have sustained release properties when administered. For water soluble actives, such as cloprostenol, a water-in-oil emulsion can provide a sustained release. In some embodiments, the composition can comprise an emulsion, or be in the form of an emulsion, or can comprise a carrier in the form of an emulsion, or can comprise a carrier comprising an emulsifier or emulsifying agent. In some embodiments, the composition or carrier can comprise an oil-in-water emulsion or a water-in-oil emulsion.

The release of an active from a water-in-oil composition can occur by several mechanisms, including partitioning from water to oil, diffusion through the oil, partitioning from the oil to the release medium (in vitro) or into the extracellular fluid (in vivo). Accordingly, the release rate can be manipulated by altering the type of oil (chemistry and physical properties, such as viscosity), additives (surfactants, gelling agents), the characteristics of the active, and water to oil ratio.

In some embodiments, the composition comprises an oily suspension of prostaglandin (such as an ester of cloprostenol) in an oily vehicle or carrier. In some embodiments, the carrier comprises at least one type of oil. An oily suspension of active is a suspension of active particles in an oily vehicle or carrier. Preferably the oily vehicle or carrier is veterinary acceptable. Such oils can include sesame oil, medium chain triglycerides such as caprylic/capric triglycerides, decanoic acid, octanoic acid, propane-1,2,3-triol (Miglyol 812), propane-1,2-diol (Miglyol 840), cottonseed oil, soybean oil, peanut oil, corn oil, and triacetin.

A gelled composition can be used to provide sustained release of an active. One method of forming a gel is by incorporating a gelling agent into a suitable vehicle or carrier that can be used to contain the active. In some embodiments, the composition can comprise a gel, or be in the form of a gel, or can comprise a carrier in the form of a gel, or can comprise a carrier comprising at least one type of gelling agent.

Another example of a gel composition is a sucrose acetate isobutyrate (SAIB) in situ gelling composition. SAIB is soluble in some water-soluble organic solvents, but has poor solubility in water. When a solution of SAIB in a water-soluble organic solvent contacts an aqueous environment, the organic solvent is diluted by the water, resulting in precipitation of the SAIB and formation of a gelled mass. The release of an active from the gelled mass is restricted (sustained). This is the basis of a so-called in situ gelling system. The active can be suspended or dissolved in the water-miscible organic solvent. The release characteristics of the formulations might be varied though choice and concentration of the water-miscible organic solvents, other additives and the characteristics of the active. Preferably the water-soluble solvent is veterinary acceptable. Suitable solvents can include ethanol, Transcutol (diethylene glycol monoethyl ether), triacetin, propylene glycol. In some embodiments, the composition comprises SAIB, or can comprise a carrier comprising SAIB. In some embodiments, the composition comprises at least one type of solvent or solvent system, or can comprise a carrier comprising at least one type of solvent or solvent system.

Compositions for injection can be prepared by dissolving or mixing the prostaglandin with a carrier. Preferably, the carrier is veterinary acceptable. The composition can be sterilised, such as by heat, filtration or irradiation, or prepared aseptically. Compositions for injection may be prepared by methods and techniques known to persons skilled in the art.

The at least one type of carrier or carrier (or additive or ingredient) can comprise one or more of the following types of veterinary acceptable ingredients/excipients/additives: a vehicle; an aqueous or oily diluent; a base; a buffering agent; a pH adjusting agent; a suspending agent; a flocculating agent; a thickener; a viscosity building agent; a gelling agent; a solvent; an organic solvent; a co-solvent; a solvent system; an emulsifier; a stabilizer; a dispersant; a detergent; a solubilizer; a fragrance; a preservative; a surfactant; an acid; a base; an antioxidant; a wetting agent; a chelating agent; a reducing agent; a bulking agent; a protectant; a tonicity adjustor; and, a colorant.

Examples of tonicity adjustors used in liquid injections or carriers include electrolytes, dextrose, glycerol, sodium chloride, glycerin and mannitol.

Examples of preservatives used in liquid injections/carriers include antioxidants, antimicrobials and chelating agents, including ascorbic acid, acetylcysteine, butylated hydroxytoluene (BHT), butylated hydroxyanisole (BHA), sulfurous acid salts (bisulfite, metabisulfite), monothioglyercol, phenol, meta-cresol, benzyl alcohol, gallate, parabens (methyl, propyl, butyl), benzalkonium chloride, chlorobutanol, thiomersal, and phenylmercuric salts.

Examples of solubilising agents used in liquid injections or carriers include surfactants, solvents and co-solvents, and include water, polyoxyethylene sorbitan monooleate (Tween 80), sorbitan monooleate polyoxyethylene sorbitan monolaurate (Tween 20), lecithin, polyoxyethylene copolymers (pluronics), propylene glycol, glycerin, organic solvents, ethanol, polyethylene glycol (300 and 400), sorbitol, dimethylacetamide and cremophor EL.

Examples of complexing and dispersing agents used in liquid injections or carriers include cyclodextrins and modified cyclodextrins such as hydroxypropyl-b-cyclodextrin and sulfobutylether-b-cyclodextrin.

Examples of buffering agents used in liquid injections or carriers include phosphate, citrate, acetate, lactate and tartrate buffers.

Suspensions may provide more sustained release of the prostaglandin from the injection site than a comparable solution. Examples of additives/ingredients/excipients/carriers used in suspensions include flocculating or suspending agents, viscosity building agents, wetting agents, solvents, solvent systems, preservatives, antioxidants, chelating agents, buffering agents, surfactants, and tonicity adjusting agents.

Examples of flocculating or suspending agents include electrolytes, surfactant and hydrophilic colloids, including potassium/sodium chloride, potassium/sodium citrate, and potassium/sodium acetate.

Examples of viscosity building agents include sodium carboxymethyl cellulose, acacia, gelatin, methyl cellulose, and polyvinyl pyrrolidone.

Examples of wetting agents include glycerin, alcohol, propylene glycol, lecithin, polysorbate 20, polysorbate 80, pluronic F-68, sorbitan, and trioleate.

Examples of solvents, including organic solvents, include water, ethanol, glycerin, propylene glycol, n-lactamide, polyethylene glycol (PEG), dimethyl sulfoxide, glycofurol, Solketal, acetone, tetrahydrofurfuryl alcohol, diglyme, dimethyl isosorbide, ethyl lactate, diethylene glycol monoethyl ether (DEGEE) (eg. sold under the trade mark Transcutol), N-methylpyrrolidone (NMP), triacetin, benzyl benzoate, miglyol, propylene carbonate, benzyl alcohol, ethyl lactate, 2-pyrrolidone, propylene glycol, methyl acetate, ethyl acetate, methyl ethyl ketone, dimethylformamide, dimethylsulfoxide, tetrahydrofuran, caprolactam, decylmethylsulfoxide, oleic acid, and 1-dodecyazacycloheptan-2-one.

In some embodiments the composition or carrier is in the form of a low viscosity, injectable gel which gels rapidly at the injection site to provide sustained release of the prostaglandin over a suitable period of time.

The composition or carrier can be made to gel in situ (in the animal) in any suitable way. In some embodiments, the composition or carrier can comprise one or more gelling agents. In some embodiments, the composition or carrier comprises one or more solvents or a solvent system, such as one or more organic solvents. In some embodiments, the composition or carrier comprises sucrose acetate isobutyrate (SAIB) and one or more solvents or a solvent system. Once a composition containing SAIB and solvent/s or solvent system is injected, the solvent/s can diffuse out leaving a matrix that is both adhesive and viscous. The matrix can retain the prostaglandin at the injection site for a prolonged period of time, rather than the prostaglandin dispersing completely almost immediately.

In some embodiments, the composition or carrier comprises SAIB in the following approximate ranges: 30% to 90%, 35% to 90%, 40% to 90%, 45% to 90%, 50% to 90%, 30% to 85%, 35% to 85%, 40% to 85%, 45% to 85%, 50% to 85%, 30% to 80%, 35% to 80%, 40% to 80%, 45% to 80%, 50% to 80%, 30% to 75%, 35% to 75%, 40% to 75%, 45% to 75%, 50% to 75%, 30% to 70%, 35% to 70%, 40% to 70%, 45% to 70%, or 50% to 70% w/v SAIB, including all numerical values between 30 to 90, including approximately 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 and 90. In some embodiments, the composition or carrier comprises about 70% to about 80% w/v SAIB. In some embodiments, the composition or carrier comprises about 70% or about 80% w/v SAIB.

Any suitable solvent/s or solvent system can be used provided that it/they produce a low viscosity, injectable gel. Preferred solvents of the composition or carrier include ethanol, diethylene glycol monoethyl ether (DEGEE) sold under the trade mark Transcutol, N-methylpyrrolidone (NMP), triacetin, benzyl benzoate, miglyol, propylene carbonate, benzyl alcohol, ethyl lactate, glycofurol, 2-pyrrolidone, propylene glycol, acetone, methyl acetate, ethyl acetate, methyl ethyl ketone, dimethylformamide, dimethylsulfoxide, tetrahydrofuran, caprolactam, decylmethylsulfoxide, oleic acid, and 1-dodecyazacycloheptan-2-one, or any mixtures of these. In some embodiments, the solvent/s of the composition or carrier can comprise a mixture of ethanol, triacetin, and diethylene glycol monoethyl ether in any suitable ratio and quantities. The solvent/s can be present in the composition or carrier in an amount of between approximately 9% to 60% w/v, preferably approximately 15 to 30% w/v. These ranges include all numerical values between 9 to 60, and 15 and 30, inclusive of about 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 and 60. In some embodiments, the solvent comprises Transcutol. In some embodiments, the solvent comprises Transcutol and triacetin. In some embodiments, the solvent comprises ethanol.

In some embodiments, the composition or carrier comprises a surfactant, detergent or emulsifier, to assist with the release of prostaglandin. Any suitable type of surfactant, detergent or emulsifier can be used. Suitable examples include non-ionic surfactants such as polysorbates. Span 80 (sorbitan monooleate, sorbitan oleate, CAS Number: 1338-43-8) and Tween 80 (POE (20) sorbitan monooleate, polyethylene glycol sorbitan monooleate, polyoxyethylenesorbitan monooleate, polysorbate 80; CAS No.: 9005-65-6) are particularly preferred. The surfactant can be present in the composition or carrier in an amount of between approximately 0.01% to 10% w/v, preferably approximately 0.01% to 0.1% w/v, and more preferably approximately 0.05% w/v. These ranges include all numerical values between 0.01 and 10, inclusive of about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95 and 0.1). In some embodiments, the composition or carrier comprises about 0.05% w/v surfactant, preferably Span 80.

In some embodiments, the composition or carrier comprises at least one type of antioxidant. Any suitable type of antioxidant or antioxidants can be used. Examples of suitable antioxidants include phenolic antioxidants, such as butylated hydroxytoluene (BHT) and butylated hydroxyanisole (BHA). BHT is particularly preferred. The antioxidant can be present in the composition or carrier in an amount of between approximately 0.01% to 10% w/v, preferably approximately 0.05% to 5% w/v. These ranges include all numerical values between 0.01 and 10, and 0.05 and 5, inclusive of about 0.01, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0 . . . , etc). In some embodiments, the composition or carrier comprises approximately 0.1% w/v antioxidant, preferably BHT.

The concentration of prostaglandin in the composition/formulation/medicament or carrier is preferably in the range of approximately 0.0001% w/v to 10% w/v depending on the potency of the prostaglandin, including all numerical values between 0.0001 and 10 (inclusive of about 0.0001 . . . 0.001 . . . 0.01 . . . 0.1 . . . 1.0 . . . 10). In some embodiments, prostaglandin is present in an amount of at least approximately 0.05% w/v. In some embodiments, prostaglandin is present in an amount of at least approximately 0.15% w/v. In some embodiments, prostaglandin is present in an amount of between approximately 0.05% to 0.5% w/v, preferably approximately 0.15% w/v, including all numerical values and sub-ranges between 0.05 and 0.5 (inclusive of about 0.05 . . . 0.1, 0.11, 0.12, 0.13, 0.14, 0.15, 0.16, 0.17, 0.18, 0.19, 0.2 . . . 0.25 . . . 0.3 . . . 0.35 . . . 0.4 . . . 0.45 . . . 0.5, and sub-ranges 0.1 and 0.2% w/v, for example). In some embodiments, the prostaglandin is PGF2α. In some embodiments, the prostaglandin is cloprostenol. In some embodiments the prostaglandin is dinoprost. In some embodiments, the composition or carrier comprises about 0.05% to 0.5% w/v of prostaglandin, PGF2α, cloprostenol or dinoprost, especially about 0.15% w/v cloprostenol (as the sodium salt).

In some embodiments, the sustained release composition comprises a prostaglandin or PGF2α, such as cloprostenol or cloprostenol sodium, in a SAIB carrier.

In some embodiments, the sustained release composition comprises a prostaglandin or PGF2α, such as dinoprost, in a SAIB carrier.

In some embodiments, the sustained release composition comprises a prostaglandin, PGF2α, cloprostenol or dinoprost, in a gel-forming carrier comprising SAIB and a water-miscible or partially water-miscible solvent/s. Such solvents comprise one or more of Transcutol, triacetin (1,2,3-triacetoxypropane, 1,2,3-triacetylglycerol, glyceryl triacetate) and ethanol.

In some embodiments, the sustained release composition comprises a prostaglandin, PGF2α, cloprostenol or dinoprost, preferably a cloprostenol ester, in a suspension comprising an oily carrier such as sesame oil.

Any suitable dose of the composition can be administered to the animal. In some embodiments, the dose may depend on the potency of the prostaglandin, the weight of the animal, or blood flow and the rate of drug metabolism. In some embodiments, approximately 0.5 to approximately 10 μg per kg bodyweight of the animal, more preferably approximately 2.5 to approximately 10 μg per kg bodyweight of the animal, and even more preferably approximately 3.3 μg per kg bodyweight of the animal is administered (inclusive of about 0.5, 1, 1.5, 2, 2.5, 3, 3.5, 4, 4.5, 5, 5.5, 6, 6.5, 7, 7.5, 8, 8.5, 9, 9.5 and 10). In some embodiments, an absolute dose is administered irrespective of the weight of the animal, such as, for example, approximately 10 μg to approximately 10 mg, preferably approximately 50 μg to approximately 5.0 mg, more preferably approximately 2.0 mg to approximately 3.0 mg (inclusive of about 0.01, 0.5, 1, 1.5, 2, 2.5, 3, 3.5, 4, 4.5, 5, 5.5, 6, 6.5, 7, 7.5, 8, 8.5, 9, 9.5 and 10 mg).

The quantity of composition administered is preferably within the range of approximately 0.5 to approximately 10 ml, including all numerical values between 0.5 and 10 (inclusive of about 0.5, 1, 1.5, 2, 2.5, 3, 3.5, 4, 4.5, 5, 5.5, 6, 6.5, 7, 7.5, 8, 8.5, 9, 9.5 and 10). A particularly preferred range is from approximately 1.0 ml to 5 ml, including all numerical values between 1.0 and 5 (inclusive of about 01, 1.5, 2, 2.5, 3, 3.5, 4, 4.5 and 5), and even more preferably up to about 2 ml. Preferably an injector is used to carry out injection of (administration to) the animal.

Particularly preferred compositions/formulations are shown below:

Formulation 1 (all % are approximate):

| Prostaglandin or PGF2α (eg. | 0.05% to 0.5% | w/v |
|---|---|---|
| cloprostenol or dinoprost) | | |
| SAIB | 80% | w/v |

| | |
|---|---|
| Solvent, such as Transcutol | quantity to suit (q.s.) |

Formulation 1A (all % are approximate):

| Prostaglandin or PGF2α, | 0.15% w/v (as | |
|---|---|---|
| such as cloprostenol | the sodium salt) | |
| SAIB | 80% | w/v |

| | |
|---|---|
| Solvent, such as Transcutol | q.s. |

Formulation 1B (all % are approximate):

| Cloprostenol | 0.15% w/v (as | |
|---|---|---|
| | the sodium salt) | |
| SAIB | 80% | w/v |

| | |
|---|---|
| Transcutol | q.s. |

Formulation 2 (all % are approximate):

| | | |
|---|---|---|
| Prostaglandin or PGF2α (eg. | 0.05% to 0.5% | w/v |
| cloprostenol or dinoprost) | | |
| SAIB | 70% | w/v |
| Partially water-miscible solvent, | 15% | w/v |
| such as Triacetin | | |

| | |
|---|---|
| Solvent, such as Transcutol | q.s. |

Formulation 2A (all % are approximate):

| Prostaglandin, such as | 0.15% w/v (as | |
|---|---|---|
| cloprostenol | the sodium salt) | |
| SAIB | 70% | w/v |
| Partially water-miscible solvent, | 15% | w/v |
| such as Triacetin | | |

| | |
|---|---|
| Solvent, such as Transcutol | q.s. |

Formulation 2B (all % are approximate):

| Cloprostenol | 0.15% w/v (as | |
|---|---|---|
| | the sodium salt) | |
| SAIB | 70% | w/v |
| Triacetin | 15% | w/v |

| | |
|---|---|
| Transcutol | q.s. |

Formulation 3 (all % are approximate):

| | | |
|---|---|---|
| Prostaglandin or PGF2α (eg. | 0.05% to 0.5% | w/v |
| cloprostenol or dinoprost) | | |
| SAIB | 70% | w/v |

| | |
|---|---|
| Solvent, such as ethanol | q.s. |

Formulation 3A

| Prostaglandin, such as | 0.15% w/v (as | |
|---|---|---|
| cloprostenol | the sodium salt) | |
| SAIB | 70% | w/v |

| | |
|---|---|
| Solvent, such as ethanol | q.s. |

Formulation 3B

| Cloprostenol | 0.15% w/v (as | |
|---|---|---|
| | the sodium salt) | |
| SAIB | 70% | w/v |

| | |
|---|---|
| Ethanol | q.s. |

Formulation 4 (all % are approximate):

| | | |
|---|---|---|
| Prostaglandin or PGF2α (eg. | 0.05% to 0.5% | w/v |
| cloprostenol or dinoprost) | | |
| Emulsifier or detergent, | 0.05% | w/v |
| such as Span 80 | | |

| | |
|---|---|
| Oil, such as sesame oil | q.s. |

Formulation 4A (all % are approximate):

| Prostaglandin, such as | 0.15% w/v (as | |
|---|---|---|
| cloprostenol | the sodium salt) | |
| Emulsifier or detergent, | 0.05% | w/v |
| such as Span 80 | | |

| | |
|---|---|
| Oil, such as sesame oil | q.s. |

Formulation 4B (all % are approximate):

| Cloprostenol | 0.15% w/v (as | |
|---|---|---|
| | the sodium salt) | |
| Span 80 | 0.05% | w/v |

| | |
|---|---|
| Sesame oil | q.s. |

A study by the authors has shown that a conventional dose of 0.5 mg of cloprostenol as the sodium salt in a fast-release aqueous composition, results in an estimated half-life of cloprostenol in plasma of 15 to 60 minutes. Literature has reported a half-life for cloprostenol of around 90 minutes, with a maximum concentration reached at around 10 to 20 minutes after administration.

Simulations were conducted to estimate the plasma profile of cloprostenol based on assuming an elimination half-life of 60 minutes, and a first order absorption from the injection site with different absorption half-lives of 10 minutes, 4 hours and 8 hours. From these simulations it was determined that a sustained release composition of cloprostenol that has a length of action of 12-24 hours will require an estimated 3 mg dose of cloprostenol, with a sustained release absorption half-life of 4 hours or more.

The inventors have also used various models to describe the absorption kinetics of cloprostenol. The most recent model was to describe the release from SAIB formulations as occurring as a burst of some percentage of the dose followed by a rate of release proportional to the square root of time. In an animal trial, the inventors calculated the terminal slope of the plasma concentration versus time profile. If absorption is very rapid—ie. as from standard cloprostenol injection, Ovuprost™, this terminal slope is related to the elimination half-life of the cloprostenol. The inventors found this half-life to be 1 hour. The literature reports the elimination half-life of cloprostenol to be 1.5-3 hours. If absorption is slow, the terminal slope relates to the absorption rate of the cloprostenol from the injection. The inventors found this half-life for the SAIB formulations to be greater than 3.5 hours.

According to Greco et al 2018, endogenous PGF2α is secreted in pulsatile fashion. In general, cows that underwent luteolysis had 2 pulses of PGF metabolite (PGFM) during the preluteolytic period, 2 pulses during the luteolytic period, and 1 pulse during the post-luteolytic period. The luteolytic period was characterized by at least 2 major PGFM pulses in most cows, however, the measures of reliability indicated that a minimum of 3 pulses of PGF2α were required to induce CL regression. The endogenous pulses of PGFM during the luteolytic period were determined by Greco as 200-300 pg/ml in plasma and about 300-600 pg/ml by McCracken, Custer, and Lamsa 1999.

Endogenous PGF2α is secreted at the uterus and transported to the ovary by the local pathway involving close apposition of efferent uterine vessels (venous and lymphatic) with ovarian arterial vessels and it has been suggested that delivery to the ovaries from the systemic circulation also occurs in cows (McCracken, Custer, and Lamsa 1999) but the effective luteolytic dose of PGF is approximately 20-fold greater when given systemically than when given into the uterus (Inskeep 1973).

PGF2α is reported to have a short half-life of about 8 minutes. Baseline PGF2α concentrations have been reported as about 80 pg/ml (Shrestha et al. 2012) and baseline PGFM concentrations are around 36-50 pg/ml. The PGFM is non-active, and so applying this ratio to the peak PGFM levels suggests that the required concentration of PGF2α in systemic blood to provide luteolytic activity can be estimated as around 400-1200 pg/ml (0.4-1.2 ng/ml). This is about 5-10× the base-line concentration.

In cattle, the conventional intramuscular injected dose of natural PGF2α is 25 mg whereas the intramuscular injected dose of cloprostenol in 0.5 mg. PGF2α has a half-life of about 8-10 minutes whereas the cloprostenol half-life is from around 1 hour as determined by the authors' study and reported elsewhere as 1.5-3 hours.

It is assumed that the affinity and efficacy of cloprostenol and PGF2α (dinoprost) for the luteal cell membrane receptors are the same; thus the target peak concentration of systemic (blood plasma) cloprostenol is also 0.4-1.2 ng/ml.

Preferably the biologically active plasma concentration of prostaglandin provided by the prolonged administration is at least about 0.1 ng/ml. For example, the approximate prolonged biologically active plasma concentration of prostaglandin provided by the prolonged administration is at least 0.1 ng/ml, at least 0.2 ng/ml, at least 0.3 ng/ml, at least 0.4 ng/ml, at least 0.5 ng/ml, at least 0.6 ng/ml, at least 0.7 ng/ml, at least 0.8 ng/ml, at least 0.9 ng/ml, at least 1.0 ng/ml, or at least 1.14 ng/ml. In some embodiments, the plasma concentration of prostaglandin provided by the prolonged administration is between about 0.1-1.2 ng/ml, more preferably about 0.4-1.2 ng/ml.

The prolonged administration of a prostaglandin can be conducted as part of a reproductive breeding programme. For example, the reproductive breeding programme can include the timed administration of GnRH and/or timed administration of progesterone.

In one reproductive breeding programme, each animal is administered GnRH, and about five days later followed by prolonged administration of a prostaglandin. Preferably the prolonged administration of a prostaglandin is the administration of a sustained release composition of PGF2α. A sustained release composition of cloprostenol or dinoprost is particularly preferred. The prolonged administration of a prostaglandin can replace the conventional administration of two separate injections of a prostaglandin 1 day apart.

In another reproductive breeding programme, a second GnRH injection is administered 3 days after the prostaglandin. In another reproductive breeding programme, progesterone is administered before a first GnRH injection.

When used herein, the term ‘GnRH’ refers to gonadotropin releasing hormone or an equivalent to endogenous gonadotropin releasing hormone, such as gonadorelin.

The present invention is discussed primarily for use with cattle, however it should be appreciated the present invention can also be used with other animal species including any type of domesticated, livestock or farm animal, including sheep, horses (eg. a mare), goats, pigs (eg. a sow) and dogs.

In some embodiments, the composition/medicament is for luteolysis of functional corpora lutea in cows, mares or sows.

### Exemplary Uses

### Dairy and Beef Cows

For use in Fixed Time Artificial Insemination programmes in cycling cows. 1 PG injection to replace 2 injections of PG.

Approximate dose: 2,000 to 3,000 μg per animal, preferably in about 2 ml, or about 3.3 to 5 μg per kg bodyweight of the animal.

Preferred: approx. 2,000 μg per animal, preferably in about 2 ml, or about 3.3 μg per kg bodyweight of the animal.

For use in treatment of anoestrus in cows. 1 PG injection to replace 2 injections of PG.

Approximate dose: 2,000 to 3,000 μg per animal, preferably in about 2 ml, or about 3.3 to 5 μg per kg bodyweight of the animal.

Preferred: approx. 2,000 μg per animal, preferably in about 2 ml, or about 3.3 μg per kg bodyweight of the animal.

### Dairy and Beef Heifers

For use in Fixed Time Artificial Insemination programmes in heifers. 1 PG injection to replace 2 injections of PG.

For use in synchrony product in heifers. 1 PG injection to replace 2 injections of PG.

Approximate dose: 2,000 to 3,000 μg per animal, preferably in about 2 ml, or about 3.3 to 5 μg per kg bodyweight of the animal.

Preferred: approx. 2,000 μg per animal, preferably in about 2 ml, or about 3.3 μg per kg bodyweight of the animal.

For use in luteolysis, synchronization or timed artificial insemination (TAI) programs, and treating anoestrus, with dosages as mentioned in the paragraphs above.

With respect to the seventh aspect of the present invention, the kit can further comprise a progesterone. In one embodiment the kit can further comprise a GnRH.

Preferred embodiments of the invention are defined in the paragraphs below:

1. A sustained release composition formulated for: inducing luteolysis of a functional corpus luteum in an animal; improving fertility in an animal; improving conception rates in a group of animals; synchronising ovulation in a group of animals; or, use in a reproductive breeding programme, wherein the composition comprises a prostaglandin.

2. Use of a prostaglandin in the manufacture of a medicament or composition for: inducing luteolysis of a functional corpus luteum in an animal; improving fertility in an animal; improving conception rates in a group of animals; synchronising ovulation in a group of animals; or, use in a reproductive breeding programme, wherein the medicament or composition provides a sustained release of the prostaglandin.

3. A sustained release composition for use or when used in a method for: inducing luteolysis of a functional corpus luteum in an animal; improving fertility in an animal; improving conception rates in a group of animals; synchronising ovulation in a group of animals; or, use in a reproductive breeding programme, wherein the composition comprises a prostaglandin.

4. A method of: inducing luteolysis of a functional corpus luteum in an animal; improving fertility in an animal; improving conception rates in a group of animals; or, synchronising ovulation in a group of animals; wherein the method comprises administering to the animal or animals a sustained release composition comprising a prostaglandin.

5. Use of a sustained release composition comprising a prostaglandin for: inducing luteolysis of a functional corpus luteum in an animal; improving fertility in an animal; improving conception rates in a group of animals; synchronising ovulation in a group of animals; or, use in a reproductive breeding programme.

6. A kit for use in: inducing luteolysis of a functional corpus luteum in an animal; improving fertility in an animal; improving conception rates in a group of animals; synchronising ovulation in a group of animals; a reproductive breeding programme; or, controlling oestrus of an animal, wherein the kit comprises a sustained release composition comprising a prostaglandin.

7. A kit for use in a method for controlling oestrus of an animal, wherein the kit comprises a sustained release composition of a prostaglandin.

8. A method of preparing a sustained release composition of a prostaglandin, said method comprising the step of mixing a therapeutic quantity of the prostaglandin with a carrier that is capable of providing the prostaglandin with a sustained release such that the terminal half-life of the plasma concentration of the prostaglandin versus time profile is approximately 3.5 hours or more.

9. The composition, method, use or kit as defined in any preceding paragraph (context permitting), wherein the prostaglandin is a prostaglandin or prostaglandin analogue, which is either naturally occurring or synthetically produced.

10. The composition, method, use or kit as defined in any preceding paragraph (context permitting), wherein the prostaglandin is Prostaglandin F2α, or an analogue thereof, including pharmaceutically acceptable salts or esters thereof, such as dinoprost, cloprostenol or cloprostenol sodium salt.

11. The composition, method, use or kit as defined in any preceding paragraph (context permitting), wherein the composition or medicament comprises or consists of: Formulation 1 (1, 1A, 1B) as described herein, Formulation 2 (2, 2A, 2B) as described herein, Formulation 3 (3, 3A, 3B) as described herein, Formulation 4 (4, 4A, 4B) as described herein, a formulation as described in Table 4, a formulation as described in Table 5, a formulation as described in Table 7, a formulation as described in Table 8, a formulation as described in Table 11, or a composition, formulation or medicament as described elsewhere in this specification.

12. The composition, method, use or kit as defined in any preceding paragraph (context permitting), for treating a condition in an animal or group of animals as described in Table 11 or elsewhere in this specification.

Further preferred embodiments of the invention are defined in the paragraphs below:

1. An injectable sustained release composition comprising a prostaglandin, formulated to release the prostaglandin in a sustained manner.

2. The composition of paragraph 1, wherein the prostaglandin is selected from prostaglandin E₁ (PGE₁), prostaglandin E₂ (PGE₂), prostaglandin F_{2α} (PGF_{2α}/PGF2α), prostacyclin (PGI₂), and thromboxane (TXA₂).

3. The composition of paragraph 2, wherein the prostaglandin is PGF2α, preferably cloprostenol or dinoprost.

4. The composition of paragraph 3, wherein the PGF2α is the synthetic analogue cloprostenol.

5. The composition of paragraph 4, wherein the cloprostenol is a cloprostenol salt such as cloprostenol sodium.

6. The composition of any one of paragraphs 1 to 5, wherein the composition comprises approximately 0.0001% w/v to 10% w/v prostaglandin such as PGF2α, or approximately 0.05% to 0.5% w/v prostaglandin such as PGF2α.

7. The composition of any one of paragraphs 1 to 6, wherein the composition is in the form of a liquid, solution, suspension, dispersion, emulsion or gel.

8. The composition of paragraph 7, wherein the composition is in the form of an injectable gel that is capable of gelling rapidly at an injection site to provide sustained release of the prostaglandin.

9. The composition of paragraph 8, wherein the composition comprises at least one gelling agent and at least one water-miscible or partially water-miscible solvent or solvent system.

10. The composition of paragraph 9, wherein the at least one gelling agent comprises sucrose acetate isobutyrate (SAIB).

11. The composition of paragraph 10, wherein the composition comprises about 70% to about 80% w/v SAIB.

12. The composition of paragraph 11, wherein the composition comprises about 70% or about 80% w/v SAIB.

13. The composition of paragraph 9, 10, 11 or 12, wherein the at least one solvent or solvent system comprises one or more of diethylene glycol monoethyl ether (Transcutol), triacetin, and ethanol.

14. The composition of paragraph 9, 10, 11, 12 or 13, wherein the composition comprises about 15% to about 30% w/v the at least one solvent or solvent system.

15. The composition of paragraph 7, wherein the composition is in the form of an injectable suspension that is capable of providing sustained release of the prostaglandin.

16. The composition of paragraph 15, wherein the composition comprises a suspension of the prostaglandin, such as a cloprostenol ester, in at least one oily carrier.

17. The composition of paragraph 16, wherein the at least one oily carrier comprises sesame oil, a medium chain triglyceride, decanoic acid, octanoic acid, propane-1,2,3-triol (Miglyol 812), propane-1,2-diol (Miglyol 840), cottonseed oil, soybean oil, peanut oil, corn oil, or triacetin.

18. The composition of paragraph 17, wherein the at least one oily carrier comprises sesame oil.

19. The composition of paragraph 16, 17 or 18, wherein the composition comprises at least one surfactant, detergent or emulsifier, preferably about 0.01% to 10% w/v.

20. The composition of paragraph 19, wherein the at least one surfactant, detergent or emulsifier comprises a polysorbate.

21. The composition of paragraph 21, wherein the polysorbate comprises sorbitan monooleate or sorbitan oleate.

22. The composition of any one of paragraphs 1 to 21, wherein the composition comprises about 0.05% to 0.5% w/v of prostaglandin, such as PGF2α, such as cloprostenol, preferably about 0.15% w/v cloprostenol (as the sodium salt).

23. The composition of paragraph 1 or any other preceding paragraph (context permitting), comprising a formulation selected from the following Formulations, wherein all ingredient percentages are approximate:

| | |
|---|---|
| Formulation 1 | |
| Prostaglandin or PGF2α | 0.05% to 0.5% w/v; |
| (eg. cloprostenol or dinoprost) | |
| SAIB | 80% w/v; and |
| Solvent, such as Transcutol | q.s., or |
| Formulation 1A | |
| Prostaglandin or PGF2α, | 0.15% w/v (as the |
| such as cloprostenol | sodium salt); |
| SAIB | 80% w/v; and |
| Solvent, such as Transcutol | q.s., or |
| Formulation 1B | |
| Cloprostenol | 0.15% w/v (as the |
| | sodium salt); |
| SAIB | 80% w/v; and |
| Transcutol | q.s., or |
| Formulation 2 | |
| Prostaglandin or PGF2α | 0.05% to 0.5% w/v; |
| (eg. cloprostenol or dinoprost) | |
| SAIB | 70% w/v; |
| Partially water-miscible | 15% w/v; and |
| solvent, such as Triacetin | |
| Solvent, such as Transcutol | q.s., or |
| Formulation 2A | |
| Prostaglandin, such as | 0.15% w/v (as the |
| cloprostenol | sodium salt); |
| SAIB | 70% w/v; |
| Partially water-miscible | 15% w/v; and |
| solvent, such as Triacetin | |
| Solvent, such as Transcutol | q.s., or |
| Formulation 2B | |
| Cloprostenol | 0.15% w/v (as the |
| | sodium salt); |
| SAIB | 70% w/v; |
| Triacetin | 15% w/v; and |
| Transcutol | q.s., or |
| Formulation 3 | |
| Prostaglandin or PGF2α | .05% to 0.5% w/v; |
| (eg. cloprostenol or dinoprost) | |
| SAIB | 70% w/v; and |
| Solvent, such as ethanol | q.s., or |
| Formulation 3A | |
| Prostaglandin, such as | 0.15% w/v (as the |
| cloprostenol | sodium salt); |
| SAIB | 70% w/v; and |
| Solvent, such as ethanol | q.s., or |
| Formulation 3B | |
| Cloprostenol | 0.15% w/v (as the |
| | sodium salt); |
| SAIB | 70% w/v; and |
| Ethanol | q.s., or |
| Formulation 4 | |
| Prostaglandin or PGF2α | 0.05% to 0.5% w/v; |
| (eg. cloprostenol or dinoprost) | |
| Emulsifier or detergent, | 0.05% w/v; and |
| such as Span 80 | |
| Oil, such as sesame oil | q.s., or |
| Formulation 4A | |
| Prostaglandin, such as | 0.15% w/v (as the |
| cloprostenol | sodium salt); |
| Emulsifier or detergent, | 0.05% w/v; and |
| such as Span 80 | |
| Oil, such as sesame oil | q.s., or |
| Formulation 4B | |
| Cloprostenol | 0.15% w/v (as the |
| | sodium salt); |
| Span 80 | 0.05% w/v; and |
| Sesame oil | q.s. |

24. A sustained release composition as defined in any one of paragraphs 1 to 23, formulated for: inducing luteolysis of a functional corpus luteum in an animal; improving fertility in an animal; improving conception rates in a group of animals; synchronising ovulation in a group of animals; or, use in a reproductive breeding programme.

25. Use of a sustained release composition as defined in any one of paragraphs 1 to 23 in the manufacture of a medicament for: inducing luteolysis of a functional corpus luteum in an animal; improving fertility in an animal; improving conception rates in a group of animals; synchronising ovulation in a group of animals; or, use in a reproductive breeding programme.

26. The sustained release composition as defined in any one of paragraphs 1 to 23, for use or when used in a method for: inducing luteolysis of a functional corpus luteum in an animal; improving fertility in an animal; improving conception rates in a group of animals; synchronising ovulation in a group of animals; or, use in a reproductive breeding programme.

27. A method of: inducing luteolysis of a functional corpus luteum in an animal; improving fertility in an animal; improving conception rates in a group of animals; or, synchronising ovulation in a group of animals; wherein the method comprises administering to the animal or animals the sustained release composition as defined in any one of paragraphs 1 to 23.

28. Use of the sustained release composition as defined in any one of paragraphs 1 to 23 for: inducing luteolysis of a functional corpus luteum in an animal; improving fertility in an animal; improving conception rates in a group of animals; synchronising ovulation in a group of animals; or, use in a reproductive breeding programme.

29. A kit for use in: inducing luteolysis of a functional corpus luteum in an animal; improving fertility in an animal; improving conception rates in a group of animals; synchronising ovulation in a group of animals; a reproductive breeding programme; or, controlling oestrus of an animal, wherein the kit comprises the sustained release composition as defined in any one of paragraphs 1 to 23.

30. A kit for use in a method for controlling oestrus of an animal, wherein the kit comprises the sustained release composition as defined in any one of paragraphs 1 to 23.

31. The composition of paragraph 24, the use of paragraph 25, the composition of paragraph 26, the method of paragraph 27, or the use of paragraph 28, wherein the animal or group of animals are domesticated, livestock or farm animals, preferably sheep, horses, goats, pigs or dogs.

32. The composition of paragraph 24, the use of paragraph 25, the composition of paragraph 26, the method of paragraph 27, or the use of paragraph 28, wherein the sustained release composition is formulated to provide, or provides, a biologically active concentration of the prostaglandin, preferably cloprostenol, for at least approximately 8 hours after administration, or for at least approximately 12 hours after administration.

33. The composition of paragraph 24, the use of paragraph 25, the composition of paragraph 26, the method of paragraph 27, or the use of paragraph 28, wherein the sustained release composition is capable of providing, or provides, the prostaglandin, preferably cloprostenol, with a sustained release such that the terminal half-life of the plasma concentration of the prostaglandin versus time profile is approximately 3.5 hours or more.

34. The composition of paragraph 24, the use of paragraph 25, the composition of paragraph 26, the method of paragraph 27, or the use of paragraph 28, wherein approximately 0.5 to approximately 10 μg of prostaglandin, preferably cloprostenol, per kg bodyweight of the animal, more preferably approximately 2.5 to approximately 10 μg per kg bodyweight of the animal, and even more preferably approximately 3.3 μg per kg bodyweight of the animal is administered.

35. The composition of paragraph 24, the use of paragraph 25, the composition of paragraph 26, the method of paragraph 27, or the use of paragraph 28, wherein an absolute dose of the composition is administered irrespective of the weight of the animal, preferably approximately 10 μg to approximately 10 mg, or approximately 50 μg to approximately 5.0 mg, or approximately 2.0 mg to approximately 3.0 mg.

36. The composition of paragraph 24, the use of paragraph 25, the composition of paragraph 26, the method of paragraph 27, or the use of paragraph 28, wherein the quantity of composition administered is within the range of approximately 0.5 to approximately 10 ml.

37. The composition of paragraph 24, the use of paragraph 25, the composition of paragraph 26, the method of paragraph 27, or the use of paragraph 28, wherein the biologically active plasma concentration of prostaglandin, preferably cloprostenol or dinoprost, provided by the prolonged administration is at least about 0.1 ng/ml, or between about 0.1-1.2 ng/ml, more preferably about 0.4-1.2 ng/ml.

38. The composition of paragraph 24, the use of paragraph 25, the composition of paragraph 26, the method of paragraph 27, or the use of paragraph 28, wherein the reproductive breeding programme further comprises the timed administration of GnRH and/or timed administration of progesterone.

39. The composition of paragraph 38, the use of paragraph 38, or the method of paragraph 38, wherein for the reproductive breeding programme each animal is administered a first GnRH, and about five days later followed by prolonged administration of prostaglandin, preferably cloprostenol, using the sustained release composition.

40. The composition of paragraph 39, the use of paragraph 39, or the method of paragraph 39, wherein a second GnRH injection is administered 3 days after the prostaglandin.

41. The composition of paragraph 39, the use of paragraph 39, or the method of paragraph 39, wherein progesterone is administered before the first GnRH injection.

42. The composition of paragraph 39, the use of paragraph 39, or the method of paragraph 39, wherein progesterone is administered before the first GnRH injection.

43. The composition of paragraph 24, the use of paragraph 25, the composition of paragraph 26, the method of paragraph 27, or the use of paragraph 28, for luteolysis of functional corpora lutea in cows, mares or sows.

44. The composition of paragraph 24, the use of paragraph 25, the composition of paragraph 26, the method of paragraph 27, or the use of paragraph 28:
for use in Fixed Time Artificial Insemination programmes in cycling cows, with the approximate dose being 2,000 to 3,000 μg per animal, preferably in about 2 ml, or about 3.3 to 5 μg per kg bodyweight of the animal; or, the approximate dose being 2,000 μg per animal, preferably in about 2 ml, or about 3.3 μg per kg bodyweight of the animal; or
for use in the treatment of anoestrus in cows, with the approximate dose being 2,000 to 3,000 μg per animal, preferably in about 2 ml, or about 3.3 to 5 μg per kg bodyweight of the animal; or, the approximate dose being 2,000 μg per animal, preferably in about 2 ml, or about 3.3 μg per kg bodyweight of the animal; or
for use in Fixed Time Artificial Insemination programmes in heifers; or
for use in synchrony product in heifers, with the approximate dose being 2,000 to 3,000 μg per animal, preferably in about 2 ml, or about 3.3 to 5 μg per kg bodyweight of the animal; or, the approximate dose being 2,000 μg per animal, preferably in about 2 ml, or about 3.3 μg per kg bodyweight of the animal.

45. A treatment protocol selected from the following:

| Formulation | Dose per animal | Treatment |
|---|---|---|
| 1, 1A, 1B, | 2.5-10 μg/kg | Inducing luteolysis of a |
| 2, 2A, 2B, | body weight | functional corpus luteum; |
| 3, 3A, 3B, | | |
| 4, 4A or 4B | | |
| 1, 1A, 1B, | 2.5-10 μg/kg | Improving fertility; |
| 2, 2A, 2B, | body weight | |
| 3, 3A, 3B, | | |
| 4, 4A or 4B | | |
| 1, 1A, 1B, | 2.5-10 μg/kg | Improving conception |
| 2, 2A, 2B, | body weight | rates in a group of animals; |
| 3, 3A, 3B, | | |
| 4, 4A or 4B | | |
| 1, 1A, 1B, | 2.5-10 μg/kg | Oestrus synchronisation |
| 2, 2A, 2B, | body weight | in a group of animals; |
| 3, 3A, 3B, | | |
| 4, 4A or 4B | | |
| 1, 1A, 1B, | 2.5-10 μg/kg | Fixed time artificial |
| 2, 2A, 2B, | body weight | insemination in cycling cows; |
| 3, 3A, 3B, | | |
| 4, 4A or 4B | | |
| 1, 1A, 1B, | 2.5-10 μg/kg | Treatment of anoestrus |
| 2, 2A, 2B, | body weight | in cows; |
| 3, 3A, 3B, | | |
| 4, 4A or 4B | | |
| 1, 1A, 1B, | 2.5-10 μg/kg | Fixed time artificial |
| 2, 2A, 2B, | body weight | insemination in heifers; or |
| 3, 3A, 3B, | | |
| 4, 4A or 4B | | |
| 1, 1A, 1B, | 2.5-10 μg/kg | Oestrus synchronisation |
| 2, 2A, 2B, | body weight | in heifers; |
| 3, 3A, 3B, | | |
| 4, 4A or 4B | | |

wherein each said Formulation comprises the following ingredients, and all ingredient percentages are approximate:

| | |
|---|---|
| Formulation 1 | |
| Prostaglandin or PGF2α | 0.05% to 0.5% w/v; |
| (eg. cloprostenol or dinoprost) | |
| SAIB | 80% w/v; and |
| Solvent, such as | q.s., |
| Transcutol | |
| Formulation 1A | |
| Prostaglandin or PGF2α, | 0.15% w/v |
| such as cloprostenol | (as the sodium salt); |
| SAIB | 80% w/v; and |
| Solvent, such as | q.s., |
| Transcutol | |
| Formulation 1B | |
| Cloprostenol | 0.15% w/v |
| | (as the sodium salt); |
| SAIB | 80% w/v; and |
| Transcutol | q.s., |
| Formulation 2 | |
| Prostaglandin or PGF2α | 0.05% to 0.5% w/v; |
| (eg. cloprostenol or dinoprost) | |
| SAIB | 70% w/v; |
| Partially water-miscible | 15% w/v; and |
| solvent, such as Triacetin | |
| Solvent, such as | q.s., |
| Transcutol | |
| Formulation 2A | |
| Prostaglandin, such as | 0.15% w/v |
| cloprostenol | (as the sodium salt); |
| SAIB | 70% w/v; |
| Partially water-miscible | 15% w/v; and |
| solvent, such as Triacetin | |
| Solvent, such as | q.s., |
| Transcutol | |
| Formulation 2B | |
| Cloprostenol | 0.15% w/v |
| | (as the sodium salt); |
| SAIB | 70% w/v; |
| Triacetin | 15% w/v; and |
| Transcutol | q.s., |
| Formulation 3 | |
| Prostaglandin or PGF2α | 0.05% to 0.5% w/v; |
| (eg. cloprostenol or dinoprost) | |
| SAIB | 70% w/v; and |
| Solvent, such as | q.s., |
| ethanol | |
| Formulation 3A | |
| Prostaglandin, such as | 0.15% w/v |
| cloprostenol | (as the sodium salt); |
| SAIB | 70% w/v; and |
| Solvent, such as | q.s., |
| ethanol | |
| Formulation 3B | |
| Cloprostenol | 0.15% w/v |
| | (as the sodium salt); |
| SAIB | 70% w/v; and |
| Ethanol | q.s., |
| Formulation 4 | |
| Prostaglandin or PGF2α | 0.05% to 0.5% w/v; |
| (eg. cloprostenol or dinoprost) | |
| Emulsifier or detergent, | 0.05% w/v; and |
| such as Span 80 | |
| Oil, such as sesame oil | q.s., |
| Formulation 4A | |
| Prostaglandin, such as | 0.15% w/v |
| cloprostenol | (as the sodium salt); |
| Emulsifier or detergent, | 0.05% w/v; and |
| such as Span 80 | |
| Oil, such as sesame oil | q.s., and |
| Formulation 4B | |
| Cloprostenol | 0.15% w/v |
| | (as the sodium salt); |
| Span 80 | 0.05% w/v; and |
| Sesame oil | q.s. |

46. A method of preparing the sustained release composition as defined in any one of paragraphs 1 to 23, said method comprising the step of mixing a therapeutic quantity of the prostaglandin with a carrier that is capable of providing the prostaglandin with a sustained release such that the terminal half-life of the plasma concentration of the prostaglandin versus time profile is approximately 3.5 hours or more.

The composition, method, use or kit as defined in any preceding paragraph (context permitting), wherein the composition or medicament comprises or consists of: Formulation 1, 1A or 1B as described herein or substantially as described herein, Formulation 2, 2A or 2B as described herein or substantially as described herein, Formulation 3, 3A or B as described herein or substantially as described herein, Formulation 4, 4A or 4B as described herein or substantially as described herein, a formulation as described in Table 4 or substantially as described in Table 4, a formulation as described in Table 5 or substantially as described in Table 5, a formulation as described in Table 7 or substantially as described in Table 7, a formulation as described in Table 8 or substantially as described in Table 8, a formulation as described in Table 11 or substantially as described in Table 11, or a composition, formulation or medicament as described elsewhere in this specification.

By ‘substantially as described’ or similar phrase, it is meant that the formulation can differ from that shown yet still perform the required function. The difference can be about 1, 2, 3, 4, 5, 6, 7, 8, 9 or 10% or more of the value shown.

Having broadly described the invention in its various embodiments, non-limiting examples of preferred embodiments will now be described.

### BRIEF DESCRIPTION OF THE DRAWINGS

FIG. 1 illustrates a rotating diffusion cell apparatus for use in testing the release profile of a composition;

FIG. 2 illustrates the release profile of a sesame oil suspension of cloprostenol and a cloprostenol solution;

FIG. 3 illustrates the release profile of Ovuprost™, an aqueous cloprostenol solution and an oily suspension of cloprostenol;

FIG. 4 illustrates the release profile of sesame oil emulsions of cloprostenol;

FIG. 5 illustrates the release profile of SAIB compositions of cloprostenol using ethanol; and

FIG. 6 illustrates the release profile of SAIB compositions of cloprostenol using Transcutol instead of ethanol.

### PREFERRED EMBODIMENTS

### Example 1

### Ovuprost™ Pharmacokinetic Study

An initial study was conducted of Ovuprost™ in 15-month old dairy heifers. The commercial product Ovuprost™ contains cloprostenol as the sodium salt, premixed with citrate buffered saline solution, at a concentration of 0.25 g/L of cloprostenol as the sodium salt.

The heifers weighing 230-300 kg (average ~260 kg) were treated with 2 ml Ovuprost™ by intramuscular injection. Serum samples were analysed for cloprostenol by LCMS analysis. The limit of quantification (LOQ) of the assay was 0.05 ng/ml and the limit of detection (LOD) was 0.025 ng/ml. The results of plasma concentrations of cloprostenol at the sample time points are shown in Table 1.

TABLE 1
Cloprostenol levels in ng/ml and time to maximum concentration
(Tmax), and maximum concentration (Cmax)

| Time | Animal ID | | | | | |
|---|---|---|---|---|---|---|
| (minutes) | 6 | 214 | 221 | 243 | 325 | 330 |
| 0 | <LOD | <LOD | <LOD | <LOD | <LOD | <LOD |
| 15 | 1.42 | 1.71 | 1.45 | 1.34 | 1.43 | 0.84 |
| 30 | 2.61 | 1.48 | 0.88 | 0.70 | 1.02 | 0.94 |
| 45 | 3.66 | 2.77 | 1.15 | 0.47 | 0.98 | 0.70 |
| 60 | 1.32 | 0.82 | 0.73 | 0.13 | 0.71 | 0.67 |
| 120 | 0.37 | <LOQ | 0.08 | <LOD | 0.44 | 0.12 |
| 240 | 0.9 | 0.18 | 0.06 | <LOD | 0.13 | 0.10 |
| 360 | 0.05 | <LOD | <LOQ | <LOD | 0.07 | 0.08 |
| 720 | 0.06 | <LOD | <LOD | <LOD | <LOQ | <LOD |
| 1440 | <LOD | <LOD | <LOQ | <LOD | <LOQ | <LOQ |
| Tmax (minutes) | 45 | 45 | 15 | 15 | 15 | 30 |
| Cmax | 3.66 | 2.77 | 1.45 | 1.34 | 1.43 | 0.94 |

The concentration max varies from around 0.94 to 3.66 μg/L (mean=1.93, SD=1.0, n=6) at a time point that varied from 15 to 45 minutes after administration.

### Example 2

### Release Study Apparatus

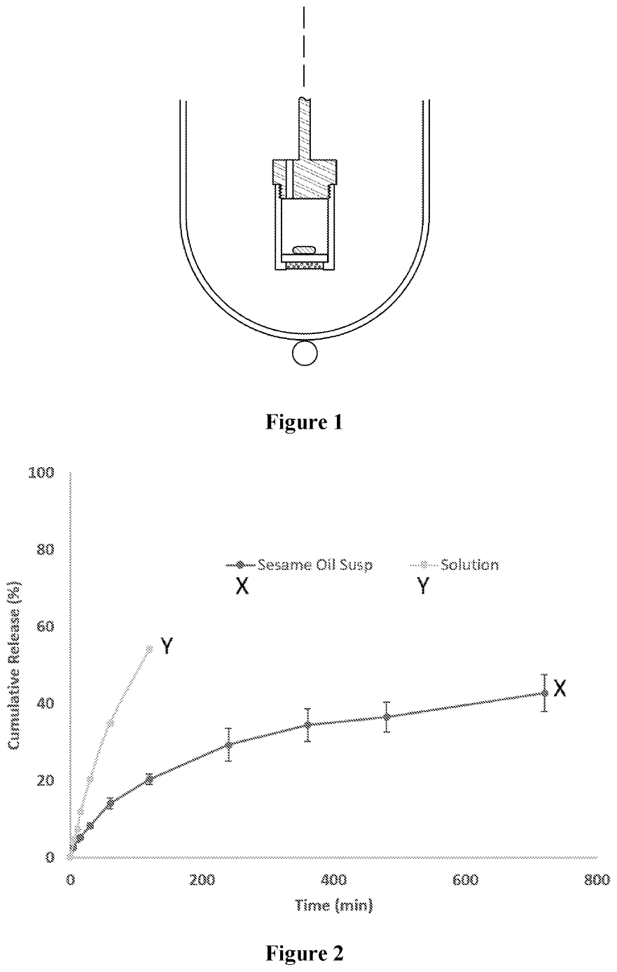

Release studies were conducted in a rotating diffusion cell as illustrated in FIG. 1. The diffusion cell is mounted in a standard USP dissolution test basket, which is a glass cylindrical vessel with a hemispherical base and a capacity of 1000 ml. The test basket is situated in a water bath maintained at 37° C.±0.5° C. The diffusion cell includes a cylindrical body and forms an internal chamber of around 3.5 ml in volume. At the top of the body is a stopper that is attached to a spindle. A conduit through the stopper provides a sampling port. A perforated gasket is situated at the base of the internal chamber. A magnetic stirrer bar rests on top of the perforated gasket. A porous membrane beneath the perforated gasket provides an exit from the diffusion cell. In use the diffusion cell is rotated via the spindle. The stirrer bar is kept from rotating by a fixed magnet beneath the test basket.

For each release study, a 0.5 g sample of composition and 0.5 g of water were pipetted into the diffusion cell which was then immersed to a standard depth (2 mm) in 400 ml of release medium (water 37° C.). The composition was separated from the release medium by the porous membrane, which in all release studies was a cellulose acetate membrane (0.2 μm pore size, 70% porosity, Advantec C020A047A). The diffusion cell was rotated at 50 rpm. The contents of the diffusion cell were mixed from the rotation of the cell relative to the stationary stirrer bar within the cell. At the predetermined times, 0.5 ml of release medium was withdrawn (with replacement) for analysis of cloprostenol by HPLC.

### Example 3

### Water-Insoluble Salt of Cloprostenol

Water-insoluble salts of cloprostenol were investigated as a means of prolonging release of cloprostenol after administration. Solutions of cloprostenol sodium were titrated with calcium chloride, zinc chloride or zinc acetate solutions. The resulting mixtures were vortexed, left to stand overnight then observed for precipitation possibly due to formation of an insoluble salt. Samples with a visible precipitate were centrifuged at 15000 rpm for 5 min and the supernatant was then removed for analysis of cloprostenol by validated HPLC.

When calcium (as calcium chloride) was used, the mixtures remained apparently clear indicating the solubility product of calcium cloprostenol had not been exceeded and that the calcium cloprostenol has good solubility.

When zinc (as zinc chloride) was used as a counter cation, immediate precipitation was observed in all samples, possibly due to formation of zinc cloprostenol. The best yield of precipitate was relatively low with the supernatant still containing around 74% of the cloprostenol.

TABLE 2
Titration of cloprostenol sodium with zinc chloride solution

| | | Molar Ratio | |
|---|---|---|---|
| Cloprostenol | | Cloprostenol/ | Cloprostenol |
| conc (M) | Zn²⁺ conc (M) | Zn²⁺ | recovery (%) |
| 0.022 | 0.015 | 1.4 | 74.6 |
| 0.022 | 0.030 | 0.7 | 78.6 |
| 0.022 | 0.152 | 0.15 | 86.4 |
| 0.022 | 1.529 | 0.015 | 88.3 |
| 0.022 | 3.05 | 0.007 | 92.5 |
| 0.022 | 6.1 | 0.003 | 82.8 |
| 0.022 | 9.1 | 0.002 | 74.6 |
| 0.022 | 12.2 | 0.001 | 78.6 |

Zinc acetate was then used to avoid issues that may have been caused by the more acidic zinc chloride. Precipitation was seen in all samples. The best yield of precipitate occurred with the supernatant still containing around 50% of the cloprostenol.

TABLE 3
Titration of cloprostenol with zinc acetate solution

| Cloprostenol | | Molar Ratio | Cloprostenol |
|---|---|---|---|
| conc (M) | Zn²⁺ conc (M) | Cloprostenol/Zn²⁺ | recovery (%) |
| 0.0005 | 0.12 | 0.004 | 58.73 |
| 0.001 | 0.12 | 0.009 | 70.68 |
| 0.002 | 0.12 | 0.018 | 84.63 |
| 0.004 | 0.12 | 0.036 | 90.41 |
| 0.009 | 0.12 | 0.072 | 49.99 |

It is assumed that multiple equilibria of different complexes are present in solution following addition of the zinc salts. Literature on the precipitation of bile acid salts with divalent cations reports that salt formation can take several weeks to reach equilibrium (Hofmann and Mysels 1992). Given the similarities in the physicochemical properties of bile acids and cloprostenol, it is possible that zinc cloprostenol complex formation is slow and that the varying recovery of cloprostenol from solution as shown in the tables is a result of the various equilibria.

### Example 4

### Oily Suspensions of Cloprostenol Sodium

Compositions of cloprostenol sodium suspended in oily vehicles/carriers were investigated. The two vehicles/carriers tested were sesame oil and Miglyol 812 (caprylic/capric triglycerides).

TABLE 4
Suspension formulations of cloprostenol in oily vehicles/carriers

| | Sesame oil | Miglyol | | |
|---|---|---|---|---|
| Ingredient | (% w/w) | (% w/w) | | |
| Cloprostenol (as sodium | 0.25 | 0.25 | 0.25 | 0.25 |
| salt) | | | | |
| Span 80 | 0.05 | 0.05 | 1.00 | — |
| Tween 80 | — | — | — | 1.00 |
| Sesame oil | QS | — | — | — |
| Miglyol 812 | — | QS | QS | QS |
| Total | 100 | 100 | 100 | 100 |

For each suspension composition, ingredients for a 20 g batch were weighed then mixed using a high-speed homogeniser (ULTRA-TURRAX, IKA Works®, T 25 B) at 24000 rpm for 3 min. Each composition was packed in a glass bottle, stored at ambient conditions and observed for physical stability every day for seven days. Release of cloprostenol was assessed at least one day after preparation.

To assess physical stability, the compositions were visually inspected for caking and adherence to the glass containers. An analysis of cloprostenol content was performed by adding to a 1 g sample of composition sufficient purified water (Milli-Q™) to bring the volume to 50 ml. The resultant mixture was bath-sonicated for 30 min and centrifuged at 14000 rpm (Eppendorf Centrifuge, 5417C) for 30 min. An aliquot of clear supernatant was diluted with ethanol (1:9) and analysis of the cloprostenol component was performed using HPLC.

The compositions of cloprostenol sodium suspended in Miglyol 812 displayed caking of the cloprostenol and the particles adhered to the walls of the glass container. Vigorous shaking was required to re-disperse the cloprostenol particles. This problem of adherence did not improve even after increasing the concentration of Span 80 (sorbitan monooleate). When Tween 80 (polysorbate 80) was included, the particles adhered to the bottom of the container and could not be re-dispersed on shaking.

The cloprostenol sodium suspended in sesame oil did not cake or adhere to the glass container, and this composition was selected for further testing.

The release properties of the sesame oil composition were assessed using the apparatus and methodology described in Example 1. An aqueous solution of cloprostenol sodium in water (1 mg/ml) was used as a control to assess the effect of the porous membrane on release. For each release study, a 0.5 g sample of composition and 0.5 g of water were pipetted into the diffusion cell, which was then immersed to a depth of 2 mm in 400 ml of water at 37° C. as a release medium. At predetermined times, 0.5 ml of release medium was withdrawn (with replacement) for analysis of cloprostenol by HPLC.

The release of cloprostenol from the sesame oil suspension and release of cloprostenol from aqueous solution were first tested with the diffusion cell rotating at 200 rpm. The results are shown in FIG. 2. The release of cloprostenol from the sesame oil suspension was significantly slower than the release from aqueous solution. The results in FIG. 2 include two tests of cloprostenol release from sesame oil, with both samples taken from the same batch of composition.

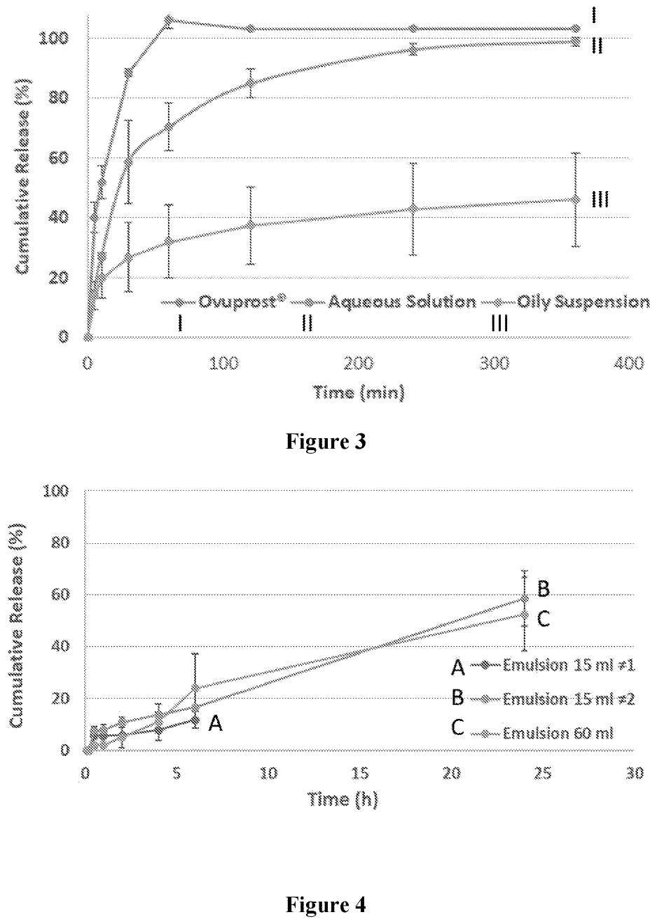

Using the same apparatus, release studies were performed with the sesame oil suspension, a 1 mg/ml aqueous solution of cloprostenol and the commercial product Ovuprost™. In these release studies the diffusion cell was rotated at 50 rpm. The results are shown in FIG. 3. The slower rotational speed resulted in a faster release rate. A relatively fast rotational speed such as 200 rpm speed may cause the formulation to centrifuge up onto the walls of the diffusion cell resulting in the slower release rate. The slower speed of 50 rpm is preferred since it is more likely to simulate intramuscular conditions.

Although not shown, at 24 h approximately 60% of cloprostenol was released from the oily suspension compared with 100% cloprostenol release at 4 h from the solution and 1-1.5 h from the Ovuprost™. Release from the two batches of the sesame oil suspension compared favorably.

The different release rates of the aqueous solution and the Ovuprost™ are possibly due to the different concentrations (aqueous solution 1 mg/ml; Ovuprost™ 0.5 mg/ml) and PH levels (aqueous solution pH ~8; Ovuprost™ PH ~5).

### Example 5

### Water-In-Oil Emulsions of Cloprostenol

Water-in-oil (w/o) emulsions of cloprostenol sodium were investigated for use as sustained release compositions of cloprostenol. The water solubility of cloprostenol sodium is greater than 10 mg/ml and is estimated to be around 35 mg/ml. In aqueous solution cloprostenol sodium micellises at concentrations around 5-10 mg/ml. The minimum quantity of water to dissolve a 3 mg dose of cloprostenol (as the sodium salt) is around 0.1 ml. Accordingly, a target volume of 2 ml of water-in-oil emulsion requires a minimum proportion of around 5% water to incorporate a 3 mg dose of cloprostenol.

Choices of emulgent and their concentrations were guided by standard HLB theory and calculations. The composition details are shown in Table 5.

TABLE 5
Water-in-oil cloprostenol compositions

| | | |
|---|---|---|
| | Sesame oil | Miglyol |
| Ingredient | (% w/w) | (% w/w) |

| | | | |
|---|---|---|---|
| Cloprostenol (as sodium salt) | 0.15 | 0.15 | 0.15 |
| Span 80 | 5 | 10 | 5 |
| Water | 20 | 25 | 20 |
| Sesame oil | QS | — | — |
| Miglyol 812 | — | QS | QS |
| Total | 100 | 100 | 100 |

The emulsions were prepared in 10 ml and 60 ml batch sizes. Sesame oil was mixed with Span 80 in a beaker by shaking for 15 min. Cloprostenol sodium was dissolved in water then mixed with Span 80. The aqueous phase was added into the oil phase and then mixed using a high-speed homogeniser (ULTRA-TURRAX, IKA Works®, T 25 B) at 19000/min for 5 min for 10 ml batches and 15 min for 60 ml batches at a duty cycle of 2 min on and 1 min off. All of the compositions formed a white foamy liquid following homogenisation.

Analysis of cloprostenol content was performed by adding to a 1 g sample of composition sufficient purified water (Milli-Q™) to bring the volume to 50 ml. The resultant mixture was bath-sonicated for 30 min and centrifuged at 14000 rpm (Eppendorf Centrifuge, 5417C) for 30 min. An aliquot of clear supernatant was diluted with ethanol (1:9) and analysis of the cloprostenol component was performed using HPLC.

The emulsions were characterized by conductivity and dye/microscopy to determine whether they were w/o or o/w emulsions, and to assess droplet size. Only the sesame oil emulsion showed spherical shaped blue droplets with no conductivity indicating a water-in-oil emulsion had been formed. The droplets had a diameter of about 50 μm.

Physical stability was screened by stressing the emulsions through centrifuging and then observing them for creaming and cracking. The formulations were then stored at ambient conditions and observed over one week. The sesame oil emulsion showed no creaming even after 1 week of storage, whilst the other emulsions separated into oil and water phases after 24 hours.

The release studies were carried out in the rotating diffusion cell using the same conditions as for the oily suspension compositions of Example 4. In addition, a mass balance assessment was conducted as follows. After the final sample time, the diffusion cell was dismantled and washed with 100 ml of water. The resultant mixture was bath-sonicated for 30 min then centrifuged at 14000 rpm (Eppendorf Centrifuge, 5417C) for 30 min and the supernatant analysed as above for content of cloprostenol.

The release studies showed 50-60% drug release after 24 h for both the small and large batches, as shown in FIG. 4. The release data percentages are means±SD, for too studies of two separately prepared 15 ml batches, and two studies of a single 60 ml batch.

The release rate from the o/w compositions was slow compared with the release rate from cloprostenol aqueous solution and Ovuprost™. To determine if any cloprostenol had not been lost during the release study, the cloprostenol remaining in the rotating diffusion cell was recovered and analysed to check the mass balance. The mass accounted for (i.e. mass released plus mass remaining in the diffusion cell) was close to 100% as shown in Table 6.

TABLE 6
Mass balance assessment

| Emulsion | Total drug content (%) | n |
|---|---|---|
| 15 ml ≠ 1 | 109 ± 2 | 2 |
| 15 ml ≠ 2 | 109 ± 3 | 2 |
| 60 ml | 100 ± 2 | 4 |

The sesame oil water-in-oil emulsion was shown to be physically and chemically stable and provide a sustained release of cloprostenol.

### Example 6

### Sucrose Acetate Isobutyrate Compositions

Sucrose acetate isobutyrate (SAIB) in situ gelling compositions of cloprostenol sodium were investigated for use as sustained release compositions of cloprostenol.

SAIB formulations were prepared using three water-miscible or partially water-miscible solvents: ethanol, triacetin, and Transcutol™ (diethylene glycol monoethyl ether).

The compositions were prepared by weighing the required amount of SAIB (sucrose acetate isobutyrate) into a glass container and adding 0.3 ml of cloprostenol solution (10 mg/ml in either ethanol or Transcutol (diethylene glycol monoethyl ether) of cloprostenol as the sodium salt). The mixture was warmed at 50° C. for 30 min before mixing by a shaker to produce a homogenous solution. The final weight was adjusted to 2 g with the selected fully/partially water-miscible solvent. The final compositions comprised 1.5 mg/g of cloprostenol (as the sodium salt). In one formulation, sodium chloride was also included.

TABLE 7
SAIB formulations using three fully/partially water-miscible organic solvents

| | % w/w in ethanol as | | | | | | | % w/w in Transcutol as | | | | |
|---|---|---|---|---|---|---|---|---|---|---|---|---|
| Ingredients | a Vehicle/Carrier | | | | | | | a Vehicle/Carrier | | | | |
| Cloprostenol as | 0.15 | 0.15 | 0.15 | 0.15 | 0.15 | 0.15 | 0.15 | 0.15 | 0.15 | 0.15 | 0.15 | 0.15 |
| the Sodium Salt | | | | | | | | | | | | |
| SAIB | 90 | 80 | 70 | 60 | 60 | 50 | 40 | 80 | 70 | 70 | 60 | 60 |
| Ethanol | 9.85 | 19.85 | 14.85 | 34.85 | 39.85 | 49.85 | 59.85 | | | | | |
| Triacetin | | | 15 | | | | | | 15 | | 20 | |
| Transcutol | | | | | | | | 19.85 | 14.85 | 29.85 | 19.85 | 39.85 |
| NaCl | | | | 5 | | | | | | | | |
| Total | 100 | 100 | 100 | 100 | 100 | 100 | 100 | 100 | 100 | 100 | 100 | 100 |

Each SAIB formulation was withdrawn manually via a 21 gauge needle and subsequently injected to observe the ease of syringeability and ability of formulation to pass through the needle. All the SAIB formulations except the formulation comprising of 90% SAIB in ethanol displayed acceptable injectability. Formulations with acceptable injectability were tested for the in-vitro release.

The chemical stability of cloprostenol was tested in ethanol and Transcutol. A cloprostenol solution (1 mg/ml) was prepared in each solvent and stored in at 40° C. for 4 days. The solutions were then removed and injected into HPLC for cloprostenol quantification and the chromatogram inspected for the formation of new (degradation) peaks. Cloprostenol in solution in ethanol and Transcutol at 40° C. was stable for the 4 days tested. The measured drug content was 101 and 102% in ethanol and Transcutol respectively. There were no degradation peaks in the chromatograms.

Initially, the release rate of the SAIB compositions was studied using the rotating diffusion cell as described in Example 1; however no drug was detected even after 8 h of study, possibly due to the blockage of the porous membrane pores by the viscous gel formed in-situ. Therefore, the release studies of the SAIB compositions were conducted using standard USP II dissolution apparatus.

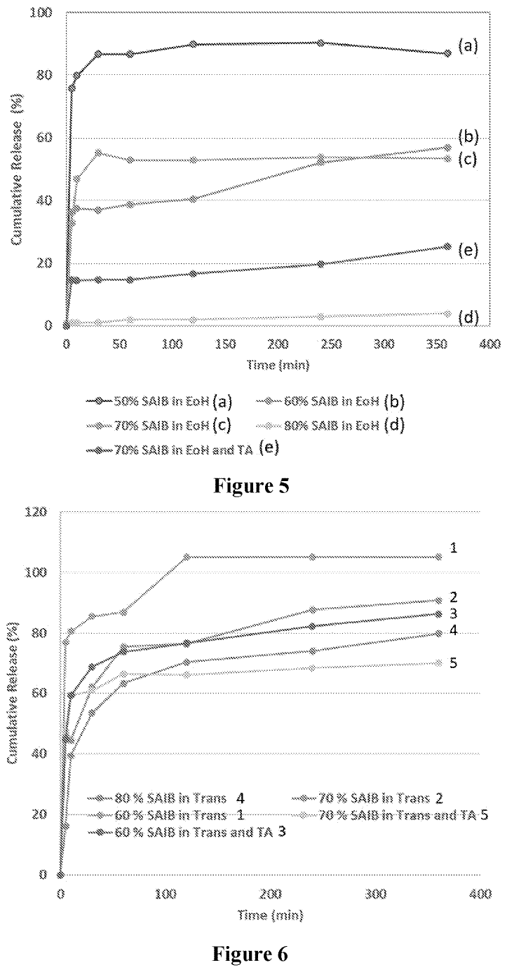

The release rate of cloprostenol from the ethanol formulations is shown in FIG. 5. The composition comprising 80% SAIB had a very slow release with only 5% of cloprostenol being released at 6 h. The composition with 50% SAIB resulted in approximately 90% of cloprostenol being released at a burst in 30 min. For all the remaining formulations, there was an initial burst release followed by a steady profile. The initial burst release was much lower in the composition including 15% triacetin. The formulation containing 40% SAIB in ethanol did not form a gel when injected into the dissolution media. Instead, the SAIB precipitated when injected and so the samples collected during release study of this composition were not analysed.

A composition including NaCl was prepared to study whether the presence of a water soluble salt in the gel would enhance the release of cloprostenol following the initial burst release. However, the compositions containing NaCl caked, and were not studied further.

As an alternative to ethanol, Transcutol (diethylene glycol monoethyl ether; 2-(2-ethoxyethoxy) ethanol) was selected for its water miscibility and its ability to dissolve SAIB to produce injectable solutions. The release profiles for the Transcutol compositions are shown in FIG. 6. Compositions having only 60% SAIB in Transcutol exhibited an 80% burst release. Increasing the SAIB to 80% prevented the burst release. Intermediate release profiles were achieved by varying the percentage of SAIB and using Transcutol alone or Transcutol and triacetin. Unlike the ethanol-based compositions, the Transcutol compositions (other than the 70% SAIB in Transcutol and triacetin) showed continuous sustained release after the initial burst release.

Cloprostenol compositions based on SAIB were shown to be stable and able to provide a sustained release of cloprostenol.

### Example 7

### Pharmacokinetic Study

A pharmacokinetic study was performed using steers to assess the performance of three classes of sustained release cloprostenol compositions. The assessed compositions were an oily suspension, a water-in-oil emulsion, and a SAIB in situ gel. The contents of the test compositions are detailed in Table 8. The animals in the study were Holstein steers. The animals were divided into four groups, with three animals per group. The animals in each of three groups were administered a test composition, and the animals in the fourth group were administered Ovuprost™ as a control.

The test compositions were administered as intramuscular injection, at a volume of 5 ml (7.5 mg of cloprostenol as the sodium salt). This is higher than the expected required dose of around 2 ml (3 mg of cloprostenol). The Ovuprost™ control was administered as an intramuscular injection of 2 ml volume (0.5 mg cloprostenol as the sodium salt).

TABLE 8
Test compositions for pharmacokinetic study

| | % w/v |
|---|---|
| SAIB 800 | |
| Cloprostenol | 0.15 |
| SAIB | 80 |
| Transcutol | q.s. |
| SAIB 700 | |
| Cloprostenol | 0.15 |
| SAIB | 70 |
| Triacetin | 15 |
| Transcutol | q.s. |
| Suspension | |
| Cloprostenol | 0.15 |
| Span 80 | 0.05 |
| Sesame oil | q.s. |
| Emulsion | |
| Cloprostenol | 0.15 |
| Span 80 | 5 |
| Water | 20 |
| Sesame oil | q.s. |

Preliminary data of the cloprostenol content in blood plasma for each animal in the study at 4 hours and 8 hours after treatment are shown in Table 9.

TABLE 9
Cloprostenol concentration in plasma

Cloprostenol concentration (ng/ml)

| Composition | 4 h | 8 h |
|---|---|---|
| Ovuprost ™ | 0.022 | 0.003 |
| Ovuprost ™ | 0.012 | 0.003 |
| Ovuprost ™ | NA | NA |
| SAIB (800) | 0.334 | 0.110 |
| SAIB (800) | 0.306 | 0.245 |
| SAIB (800) | 0.266 | 0.171 |
| SAIB (700) | 0.298 | 0.073 |
| SAIB (700) | 0.253 | 0.099 |
| SAIB (700) | 0.503 | 0.225 |
| Suspension | 0.539 | 0.241 |
| Suspension | 0.331 | 0.134 |
| Suspension | 0.196 | 0.052 |
| Emulsion | 0.170 | 0.034 |
| Emulsion | NA | 0.032 |
| Emulsion | NA | NA |

An analysis of the results is shown in Table 10. The dose correction for each time point is taken by dividing the mean plasma concentration by the dose of cloprostenol administered.

At the 4 h time point the dose-corrected plasma concentration of cloprostenol for the SAIB and suspension formulations are higher than for the Ovuprost™ control, and the dose-corrected result for the emulsion formulation is lower than the control.

The comp/control ratio is determined by dividing the dose-corrected value for the tested composition by the dose-corrected value for the control at the selected time point.

TABLE 10
Cloprostenol concentration in plasma for test compositions and control

| | | | Dose | Comp/ | | Dose | Comp/ |
|---|---|---|---|---|---|---|---|
| Dose | | 4 h | correction | Control | 8 h | correction | Control |
| (mg) | | mean | (mean/mg) | 4 h | mean | (mean/mg) | 8 h |
| 0.5 | Ovuprost ™ | 0.017 | 0.034 | — | 0.003 | 0.006 | — |
| 7.5 | SAIB (800) | 0.302 | 0.040 | 1.18 | 0.175 | 0.023 | 3.90 |
| 7.5 | SAIB (700) | 0.351 | 0.047 | 1.38 | 0.132 | 0.018 | 2.94 |
| 7.5 | Suspension | 0.355 | 0.047 | 1.39 | 0.142 | 0.019 | 3.16 |
| 7.5 | Emulsion | 0.170 | 0.023 | 0.67 | 0.033 | 0.004 | 0.73 |

The preliminary data indicates that the SAIB and suspension formulations are providing a sustained administration of cloprostenol. The tested emulsion composition is not showing a sustained release of cloprostenol, possibly because it is releasing the active too rapidly at the time of administration, or the active is trapped at the injection site.

Once all samples have been analysed the areas under curve (AUC) and relative bioavailabilities of the compositions will be determined.

### Example 8

### Sustained Release Cloprostenol Compositions for Use in Reproductive Protocols

Based on the findings in the earlier Examples, sustained release compositions formulated for use in reproductive protocols are summarised in Table 11 below.

TABLE 11
Sustained release cloprostenol compositions
for use in reproductive protocols

| | | Examples of Reproductive |
|---|---|---|
| Formulation | | Uses (examples only, not |
| (See Table 8) | Dose | exclusive/exhaustive) |
| SAIB (800) | 2.5-10 μg/kg | Inducing luteolysis of a |
| | body weight | functional corpus luteum. |
| SAIB (700) | 2.5-10 μg/kg | |
| | body weight | |
| Suspension | 2.5-10 μg/kg | |
| | body weight | |
| SAIB (800) | 2.5-10 μg/kg | Improving fertility. |
| | body weight | |
| SAIB (700) | 2.5-10 μg/kg | |
| | body weight | |
| Suspension | 2.5-10 μg/kg | |
| | body weight | |
| SAIB (800) | 2.5-10 μg/kg | Improving conception rates |
| | body weight | in a group of animals. |
| SAIB (700) | 2.5-10 μg/kg | |
| | body weight | |
| Suspension | 2.5-10 μg/kg | |
| | body weight | |
| SAIB (800) | 2.5-10 μg/kg | Oestrus synchronisation |
| | body weight | in a group of animals. |
| SAIB (700) | 2.5-10 μg/kg | |
| | body weight | |
| Suspension | 2.5-10 μg/kg | |
| | body weight | |
| SAIB (800) | 2.5-10 μg/kg | Fixed time artificial |
| | body weight | insemination in cycling cows. |
| SAIB (700) | 2.5-10 μg/kg | |
| | body weight | |
| Suspension | 2.5-10 μg/kg | |
| | body weight | |
| SAIB (800) | 2.5-10 μg/kg | Treatment of anoestrus |
| | body weight | in cows. |
| SAIB (700) | 2.5-10 μg/kg | |
| | body weight | |
| Suspension | 2.5-10 μg/kg | |
| | body weight | |
| SAIB (800) | 2.5-10 μg/kg | Fixed time artificial |
| | body weight | insemination in heifers. |
| SAIB (700) | 2.5-10 μg/kg | |
| | body weight | |
| Suspension | 2.5-10 μg/kg | |
| | body weight | |
| SAIB (800) | 2.5-10 μg/kg | Oestrus synchronisation |
| | body weight | in heifers. |
| SAIB (700) | 2.5-10 μg/kg | |
| | body weight | |
| Suspension | 2.5-10 μg/kg | |
| | body weight | |

Aspects of the present invention have been described by way of example only and it should be appreciated that modifications and additions may be made thereto without departing from the scope thereof as defined in the appended claims.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

The terms “about” and “approximately” denote an interval of accuracy that a person skilled in the art will understand to still ensure the technical effect of the feature in question. The term typically indicates a deviation from the indicated numerical value of ±10%, preferably ±5%, more preferably ±2%, and even more preferably ±1%.

Reference in this specification to any number range includes all possible numbers/numerical values falling within that range, and further includes all possible subranges falling within that range, context permitting. The notation “ . . . ” indicates that, although not shown, all relevant numbers/numerical values are to be included.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

1. An injectable sustained release composition comprising a prostaglandin, formulated to release the prostaglandin in a sustained manner, wherein the prostaglandin is selected from the group consisting of prostaglandin E₁ (PGE₁), prostaglandin E₂ (PGE₂), prostaglandin F_{2α} (PGF2α/PGF2α), prostacyclin (PGI₂), and thromboxane (TXA₂).

2. The composition of claim 1, wherein the prostaglandin is PGF2α.

3. The composition of claim 2, wherein the PGF2α is selected from the group consisting of cloprostenol and dinoprost.

4. (canceled)

5. The composition of claim 3, wherein the cloprostenol is selected from the group consisting of a cloprostenol salt, cloprostenol sodium, and anhydrous cloprostenol sodium.

6. The composition of claim 1, wherein the composition comprises a concentration selected from the group consisting of approximately 0.0001% w/v to 10% w/v prostaglandin, 0.0001% w/v to 10% w/v PGF2α, 0.05% to 0.5% w/v prostaglandin, and 0.05% to 0.5% w/v PGF2α.

7. The composition of claim 1, wherein the composition is in the form of an in situ gelling composition.

8. (canceled)

9. The composition of claim 7, wherein the composition comprises at least one gelling agent and at least one water-miscible or partially water-miscible solvent or solvent system.

10. The composition of claim 9, wherein the at least one gelling agent comprises sucrose acetate isobutyrate (SAIB).

11. The composition of claim 10, wherein the composition comprises approximately 70% to approximately 85% w/v SAIB.

12. The composition of claim 11, wherein the composition comprises approximately 80% w/v SAIB.

13. The composition of claim 9, wherein the at least one solvent or solvent system comprises one or more solvents selected from the group consisting of diethylene glycol monoethyl ether, triacetin, and ethanol.

14. The composition of claim 13, wherein the composition comprises approximately 15% to approximately 30% w/v of the at least one solvent or solvent system.

15. The composition of claim 14, wherein the at least one solvent or solvent system comprises ethanol.

16.-22. (canceled)

23. The composition of claim 1, comprising a formulation selected from the following group of formulations, wherein all ingredient percentages are approximate:

| | |
|---|---|
| Prostaglandin | 0.05% to 0.5% w/v; |
| SAIB | 80% w/v; and |
| Solvent | quantity to suit; |
| or | |
| Prostaglandin | 0.05% to 0.5% w/v; |
| SAIB | 80% w/v; and |
| Ethanol as the solvent | quantity to suit; |
| or | |
| PGF2α | 0.05% to 0.5% w/v; |
| SAIB | 80% w/v; and |
| Solvent | quantity to suit; |
| or | |
| PGF2α | 0.05% to 0.5% w/v; |
| SAIB | 80% w/v; and |
| Ethanol as the solvent | quantity to suit; |
| or | |
| Cloprostenol | 0.05% to 0.5% w/v; |
| SAIB | 80% w/v; and |
| Solvent | quantity to suit; |
| or | |
| Cloprostenol | 0.05% to 0.5% w/v; |
| SAIB | 80% w/v; and |
| Ethanol as the solvent | quantity to suit; |
| or | |
| Cloprostenol, added as a sodium salt | 0.05% to 0.5% w/v; |
| SAIB | 80% w/v; and |
| Solvent | quantity to suit; |
| or | |
| Cloprostenol, added as a sodium salt | 0.05% to 0.5% w/v; |
| SAIB | 80% w/v; and |
| Ethanol as the solvent | quantity to suit; |
| or | |
| Prostaglandin | 0.15% w/v; |
| SAIB | 80% w/v; and |
| Solvent | quantity to suit; |
| or | |
| Prostaglandin | 0.15% w/v; |
| SAIB | 80% w/v; and |
| Ethanol as the solvent | quantity to suit; |
| or | |
| PGF2α | 0.15% w/v; |
| SAIB | 80% w/v; and |
| Solvent | quantity to suit; |
| or | |
| PGF2α | 0.15% w/v; |
| SAIB | 80% w/v; and |
| Ethanol as the solvent | quantity to suit; |
| or | |
| Cloprostenol | 0.15% w/v; |
| SAIB | 80% w/v; and |
| Solvent | quantity to suit; |
| or | |
| Cloprostenol | 0.15% w/v; |
| SAIB | 80% w/v; and |
| Ethanol as the solvent | quantity to suit; |
| or | |
| Cloprostenol, added as a sodium salt | 0.15% w/v; |
| SAIB | 80% w/v; and |
| Solvent | quantity to suit; |
| or | |
| Cloprostenol, added as a sodium salt | 0.15% w/v; |
| SAIB | 80% w/v; and |
| Ethanol as the solvent | quantity to suit; |
| or | |
| Prostaglandin | 0.1% w/v; |
| SAIB | 80% w/v; and |
| Solvent | quantity to suit; |
| or | |
| Prostaglandin | 0.1% w/v; |
| SAIB | 80% w/v; and |
| Ethanol as the solvent | quantity to suit; |
| or | |
| PGF2α | 0.1% w/v; |
| SAIB | 80% w/v; and |
| Solvent | quantity to suit; |
| or | |
| PGF2α | 0.1% w/v; |
| SAIB | 80% w/v; and |
| Ethanol as the solvent | quantity to suit; |
| or | |
| Cloprostenol | 0.1% w/v; |
| SAIB | 80% w/v; and |
| Solvent | quantity to suit; |
| or | |
| Cloprostenol | 0.1% w/v; |
| SAIB | 80% w/v; and |
| Ethanol as the solvent | quantity to suit; |
| or | |
| Cloprostenol, added as a sodium salt | 0.1% w/v; |
| SAIB | 80% w/v; and |
| Solvent | quantity to suit; |
| or | |
| Cloprostenol, added as a sodium salt | 0.1% w/v; |
| SAIB | 80% w/v; and |
| Ethanol as the solvent | quantity to suit. |

24.-46. (canceled)

47. The composition of claim 23, comprising a formulation selected from the following group of formulations, wherein all ingredient percentages are approximate:

| | |
|---|---|
| Cloprostenol | 0.15% w/v; |
| SAIB | 80% w/v; and |
| Ethanol as the solvent | quantity to suit; |
| or | |
| Cloprostenol | 0.1% w/v; |
| SAIB | 80% w/v; and |
| Ethanol as the solvent | quantity to suit; |
| or | |
| Cloprostenol | 0.1% to 0.2% w/v; |
| SAIB | 80% w/v; and |
| Ethanol as the solvent | quantity to suit. |

48. A method of: inducing luteolysis of a functional corpus luteum in an animal; improving fertility in an animal; improving conception rates in a group of animals; or, synchronising ovulation in a group of animals; wherein the method comprises administering to the animal or animals the sustained release composition as defined in claim 1.

49. A method of: inducing luteolysis of a functional corpus luteum in an animal; improving fertility in an animal; improving conception rates in a group of animals; or, synchronising ovulation in a group of animals; wherein the method comprises administering to the animal or animals the sustained release composition as defined in claim 47.

50. The method of claim 48, comprising a property or step selected from the group consisting of:
the sustained release composition provides a biologically active concentration of the prostaglandin for at least approximately 8 hours after administration;
the sustained release composition provides a biologically active concentration of the prostaglandin for at least approximately 12 hours after administration;
the sustained release composition provides the prostaglandin with a sustained release such that the terminal half-life of the plasma concentration of the prostaglandin versus time profile is approximately 3.5 hours or more;
approximately 0.5 to approximately 10 μg of prostaglandin per kg bodyweight of the animal is administered;
an absolute dose of the composition is administered irrespective of the weight of the animal, approximately 10 μg to approximately 10 mg;
the quantity of composition administered is within the range of approximately 0.5 to approximately 10 ml; and
the biologically active blood plasma concentration of prostaglandin provided by the prolonged administration is at least about 0.1 ng/ml.

51. The method of claim 48, comprising a treatment protocol selected from the following:

| Formulation | Dose per animal | Treatment |
|---|---|---|
| 1, 1A, 3, 3A, | 2.5-10 μg/kg | Inducing luteolysis of a |
| or 3B | body weight | functional corpus luteum; |
| 1, 1A, 3, 3A, | 2.5-10 μg/kg | Improving fertility; |
| or 3B | body weight | |
| 1, 1A, 3, 3A, | 2.5-10 μg/kg | Improving conception rates |
| or 3B | body weight | in a group of animals; |
| 1, 1A, 3, 3A, | 2.5-10 μg/kg | Oestrus synchronisation |
| or 3B | body weight | in a group of animals; |
| 1, 1A, 3, 3A, | 2.5-10 μg/kg | Fixed time artificial |
| or 3B | body weight | insemination in cycling cows; |
| 1, 1A, 3, 3A, | 2.5-10 μg/kg | Treatment of anoestrus in |
| or 3B | body weight | cows; |
| 1, 1A, 3, 3A, | 2.5-10 μg/kg | Fixed time artificial |
| or 3B | body weight | insemination in heifers; or |
| 1, 1A, 3, 3A, | 2.5-10 μg/kg | Oestrus synchronisation |
| or 3B | body weight | in heifers, |

wherein each said Formulation comprises the following ingredients, and all ingredient percentages are approximate:

| | |
|---|---|
| Formulation 1 | |
| Prostaglandin, PGF2α, or cloprostenol | 0.05% to 0.5% w/v; |
| SAIB | 80% w/v; and |
| Solvent | quantity to suit, |
| Formulation 1A | |
| Prostaglandin, PGF2α, or cloprostenol | 0.15% w/v, wherein cloprostenol |
| | is added as a sodium salt; |
| SAIB | 80% w/v; and |
| Solvent | quantity to suit, |
| Formulation 3 | |
| Prostaglandin, PGF2α, or cloprostenol | 0.05% to 0.5% w/v; |
| SAIB | 70% w/v; and |
| Solvent, such as ethanol | quantity to suit, |
| Formulation 3A | |
| Prostaglandin, PGF2α, or cloprostenol | 0.15% w/v, wherein cloprostenol |
| | is added as a sodium salt; |
| SAIB | 70% w/v; and |
| Solvent, such as ethanol | quantity to suit, or |
| Formulation 3B | |
| Cloprostenol | 0.15% w/v, wherein cloprostenol |
| | is added as a sodium salt; |
| SAIB | 70% w/v; and |
| Ethanol | quantity to suit. |
